SEQUENCE LISTING (33 sequences)

SEQ ID NO: 1 - protein, 118 aa, Artificial Sequence
  Artificial sequence derived from Homo sapiens sequence
GVQLLESGGGLVQPGGSLRLSCAASGFTFAWYDMGWVRQAPGKGLEWVSSIDWHGEVTYYADSVKGRFTISRDNSKNTLYLQMNSLRAEDTAVYYCATAEDEPGYDYWGQGTLVTVSS

SEQ ID NO: 2 - protein, 118 aa, Artificial Sequence
  Artificial sequence derived from Homo sapiens sequence
GVQLLESGGGLVQPGGSLRLSCAASGFVFPWYDMGWVRQAPGKGLEWVSSIDWKGGKTYYADSVKGRFTISRDNSKNTLYLQMNSLRAEDTAVYYCATAEDEPGYDYWGQGTLVTVSS

SEQ ID NO: 3 - protein, 118 aa, Artificial Sequence
  Artificial sequence derived from Homo sapiens sequence
GVQLLESGGGLVQPGGSLRLSCAASGFVFAWYDMGWVRQAPGKGLEWVSSIDWHGEVTYYADSVKGRFTISRDNSKNTLYLQMNSLRAEDTAVYYCATAEDEPGYDYWGQGTLVTVSS

SEQ ID NO: 4 - protein, 118 aa, Artificial Sequence
  Artificial sequence derived from Homo sapiens sequence
GVQLLESGGGLVQPGGSLRLSCAASGFVFAWYDMGWVRQAPGKGLEWVSSIDWHGKITYYADSVKGRFTISRDNSKNTLYLQMNSLRAEDTAVYYCATAEDEPGYDYWGQGTLVTVSS

SEQ ID NO: 5 - protein, 118 aa, Artificial Sequence
  Artificial sequence derived from Homo sapiens sequence
GVQLLESGGGLVQPGGSLRLSCAASGFVFPWYDMGWVRQAPGKGLEWVSSIDWHGKITYYADSVKGRFTISRDNSKNTLYLQMNSLRAEDTAVYYCATAEDEPGYDYWGQGTLVTVSS

SEQ ID NO: 6 - DNA, 354 bp, Artificial Sequence
  Artificial sequence derived from Homo sapiens sequence
ggggtgcagctgttggagtctgggggaggcttggtacagcctggggggtccctgcgtctctcctgtgcagcctccggattcgtgttcccgtggtatgatatggggtgggtccgccaggctccagggaagggtctagagtgggtctcaagtattgattggaagggtggtaagacatactacgcagactccgtgaagggccggttcaccatctcccgcgacaattccaagaacacgctgtatctgcaaatgaacagcctgcgtgccgaggacaccgcggtatattactgtgcgacagcggaggacgagccggggtatgactactggggtcagggaaccctggtcaccgtctcgagc

SEQ ID NO: 7 - DNA, 354 bp, Artificial Sequence
  Artificial sequence derived from Homo sapiens sequence
ggggtgcagctgttggagtctgggggaggcttggtacagcctggggggtccctgcgtctctcctgtgcagcctccggattcgtcttcgcttggtatgatatggggtgggtccgccaggctccagggaagggtctagagtgggtctcaagtattgattggcatggtgaggttacatactacgcagactccgtgaagggccggttcaccatctcccgcgacaattccaagaacacgctgtatctgcaaatgaacagcctgcgtgccgaggacaccgcggtatattactgtgcgacagcggaggacgagccggggtatgactactggggccagggaaccctggtcaccgtctcgagc

SEQ ID NO: 8 - DNA, 354 bp, Artificial Sequence
  Artificial sequence derived from Homo sapiens sequence
ggggtgcagctgttggagtctgggggaggcttggtacagcctggggggtccctgcgtctctcctgtgcagcctccggattcgtcttcgcttggtatgatatggggtgggtccgccaggctccagggaagggtctagagtgggtctcaagtattgattggcatggtaagattacatactacgcagactccgtgaagggccggttcaccatctcccgcgacaattccaagaacacgctgtatctgcaaatgaacagcctgcgtgccgaggacaccgcggtatattactgtgcgacagcggaggacgagccggggtatgactactggggtcagggaaccctggtcaccgtctctagc

SEQ ID NO: 9 - DNA, 354 bp, Artificial Sequence
  Artificial sequence derived from Homo sapiens sequence
ggggtgcagctgttggagtctgggggaggcttggtacagcctggggggtccctgcgtctctcctgtgcagcctccggattcgtgttcccgtggtatgatatggggtgggtccgccaggctccagggaagggtctagagtgggtctcaagtattgattggcatggtaagattacatactacgcagactccgtgaagggccggttcaccatctcccgcgacaattccaagaacacgctgtatctgcaaatgaacagcctgcgtgccgaggacaccgcggtatattactgtgcgacagcggaggacgagccggggtatgactactggggtcagggaaccctggtcaccgtctctagc

SEQ ID NO: 10 - DNA, 354 bp, Artificial Sequence
  Artificial sequence derived from Homo sapiens sequence
ggggtgcagctgttggagtctgggggaggcttggtacagcctggggggtccctgcgtctctcctgtgcagcctccggattcaccttcgcttggtatgatatggggtgggtccgccaggctccagggaagggtctagagtgggtctcaagtattgattggcatggtgaggttacatactacgcagactccgtgaagggccggttcaccatctcccgcgacaattccaagaacacgctgtatctgcaaatgaacagcctgcgtgccgaggacaccgcggtatattactgtgcgacagcggaggacgagccggggtatgactactggggccagggaaccctggtcaccgtctctagc

SEQ ID NO: 11 - protein, 5 aa, Artificial Sequence
  Artificial sequence derived from Homo sapiens sequence
WYDMG

SEQ ID NO: 12 - protein, 17 aa, Artificial Sequence
  Artificial sequence derived from Homo sapiens sequence
SIDWHGEVTYYADSVKG

SEQ ID NO: 13 - protein, 9 aa, Artificial Sequence
  Artificial sequence derived from Homo sapiens sequence
AEDEPGYDY

SEQ ID NO: 14 - protein, 5 aa, Artificial Sequence
  Artificial sequence derived from Homo sapiens sequence
WYDMG

SEQ ID NO: 15 - protein, 17 aa, Artificial Sequence
  Artificial sequence derived from Homo sapiens sequence
SIDWKGGKTYYADSVKG

SEQ ID NO: 16 - protein, 9 aa, Artificial Sequence
  Artificial sequence derived from Homo sapiens sequence
AEDEPGYDY

SEQ ID NO: 17 - protein, 5 aa, Artificial Sequence
  Artificial sequence derived from Homo sapiens sequence
WYDMG

SEQ ID NO: 18 - protein, 17 aa, Artificial Sequence
  Artificial sequence derived from Homo sapiens sequence
SIDWHGEVTYYADSVKG

SEQ ID NO: 19 - protein, 9 aa, Artificial Sequence
  Artificial sequence derived from Homo sapiens sequence
AEDEPGYDY

SEQ ID NO: 20 - protein, 5 aa, Artificial Sequence
  Artificial sequence derived from Homo sapiens sequence
WYDMG

SEQ ID NO: 21 - protein, 17 aa, Artificial Sequence
  Artificial sequence derived from Homo sapiens sequence
SIDWHGKITYYADSVKG

SEQ ID NO: 22 - protein, 9 aa, Artificial Sequence
  Artificial sequence derived from Homo sapiens sequence
AEDEPGYDY

SEQ ID NO: 23 - protein, 5 aa, Artificial Sequence
  Artificial sequence derived from Homo sapiens sequence
WYDMG

SEQ ID NO: 24 - protein, 17 aa, Artificial Sequence
  Artificial sequence derived from Homo sapiens sequence
SIDWHGKITYYADSVKG

SEQ ID NO: 25 - protein, 9 aa, Artificial Sequence
  Artificial sequence derived from Homo sapiens sequence
AEDEPGYDY

SEQ ID NO: 26 - protein, 4 aa, Artificial Sequence
  Artificial sequence derived from Homo sapiens sequence
HGGK

SEQ ID NO: 27 - protein, 4 aa, Artificial Sequence
  Artificial sequence derived from Homo sapiens sequence
KGGK

SEQ ID NO: 28 - protein, 4 aa, Artificial Sequence
  Artificial sequence derived from Homo sapiens sequence
HGKI

SEQ ID NO: 29 - protein, 4 aa, Artificial Sequence
  Artificial sequence derived from Homo sapiens sequence
HGKI

SEQ ID NO: 30 - protein, 4 aa, Artificial Sequence
  Artificial sequence derived from Homo sapiens sequence
KGGK

SEQ ID NO: 31 - protein, 17 aa, Artificial Sequence
  Artificial sequence derived from Homo sapiens sequence
SIDWKGGKTYYADSVKG

SEQ ID NO: 32 - protein, 17 aa, Artificial Sequence
  Artificial sequence derived from Homo sapiens sequence
SIDWHGKITYYADSVKG

SEQ ID NO: 33 - protein, 7 aa, Artificial Sequence
  Artificial sequence derived from Homo sapiens sequence
ASTKGPS

(19) United States
(12) Patent Application Publication
(10) Pub. No.: US 2011/0236380 A1
(43) Pub. Date: Sep. 29, 2011

(54) LIGANDS THAT BIND IL-13

(76) Inventors: Rudolf M. T. De Wildt, Cambridgeshire (GB); Inusha De Silva, Cambridgeshire (GB); Milan Ovecka, Cambridgeshire (GB)

(21) Appl. No.: 13/131,438

(22) PCT Filed: Dec. 17, 2008
(86) PCT No.: PCT/EP2008/067789

§ 371 Date: May 26, 2011

Related U.S. Application Data
(60) Provisional application No. 61/118,013, filed on Nov. 26, 2008.

(51) Int. Cl. A61K 39/395; C12N 15/63; C12N 5/10; C12N 1/00; C12P 21/00; C07K 16/24; C07H 21/04; C07H 21/00; C07K 16/46; C07K 19/00; A61P 11/00; A61P 11/06; A61P 35/00; A61P 37/06; A61P 11/02; A61P 37/08; A61P 31/00; A61P 31/16; A61P 11/08

(52) U.S. Cl. 424/133.1; 435/320.1; 435/328; 435/243; 435/69.6; 530/387.3; 536/23.53; 536/23.4; 530/363; 530/391.1

(57) ABSTRACT

Disclosed are ligands that have binding specificity for interleukin-13 (IL-13), or for IL-4 and IL-13. Also disclosed are methods of using these ligands. In particular, the use of these ligands for treating IL-13-mediated conditions, such as lung conditions, eg allergic asthma, is described. The ligands have potent IL-13 binding kinetics. Ligands are described that are cross-reactive between human IL-13 and one or more primate IL-13. Ligands are well expressed in prokaryotic cells.

### BACKGROUND OF THE INVENTION

Th2-type immune responses promote antibody production and humoral immunity, and are elaborated to fight off extracellular pathogens. Th2 cells are mediators of Ig production (humoral immunity) and produce IL-4, IL-5, IL-6, IL-9, IL-10 and IL-13 (Tanaka, et. al., Cytokine Regulation of Humoral Immunity, 251-272, Snapper, ed., John Wiley and Sons, New York (1996)). Th2-type immune responses are characterized by the generation of certain cytokines (e.g, IL-4, IL-13) and specific types of antibodies (IgE, IgG4) and are typical of allergic reactions, which may result in watery eyes and asthmatic symptoms, such as airway inflammation and contraction of airway muscle cells in the lungs.

Interleukin-13 (IL-13) is a pleiotropic cytokine that induces immunoglobulin isotype switching to IgG4 and IgE, CD23 up regulation, VCAM-1 expression, and directly activates eosinphils and mast cells, for example. IL-13 is mainly produced by Th2 cells and inhibits the production of inflammatory cytokines (IL-1, IL-6, TNF, IL-8) by LPS-stimulated monocytes. IL-13 is closely related to IL-4 with which it shares 20-25% sequence similarity at the amino acid level. (Minty et. al., Nature, 363(6417):248-50 (1993)). Although many activities of IL-13 are similar to those of IL-4, IL-13 does not have growth promoting effects on activated T cells or T cells clones as IL-4 does. (Zurawski et al., EMBO J. 12:2663 (1993)).

The cell surface receptors and receptor complexes bind IL-13 with different affinities. The principle components of receptors and receptor complexes that bind IL-13 are IL-4Rα, IL-13Rα1 and IL-13Rα2. These chains are expressed on the surface of cells as monomers or heterodimers of IL-4Rα/IL-13Rα1 or IL-4Rα/IL-13Rα2. IL-4rα monomer binds IL-4, but not IL-13. IL-13Rα1 and IL-13Rα2 monomers bind IL-13, but do not bind IL-4. IL-4Rα/IL-13Rα1 and IL-4Rα/IL-13Rα2 heterodimers bind both IL-4 and IL-13.

IL-13 is a therapeutically important protein based on its biological functions. IL-13 has shown the potential to enhance anti-tumor immune responses. Since IL-13 is involved in the pathogenesis of allergic diseases, inhibitors of this cytokine could provide therapeutic benefits. IL-13 inhibitors are disclosed in WO2007085815, the disclosure of which is incorporated herein by reference. WO2007085815 discloses a good anti-IL-13 antibody single variable domain, DOM10-53-474. A need exists for improved agents that inhibit IL-13, and consequently the present inventors sought to find IL-13 inhibitors that perform even better than DOM10-53-474, in particular to find inhibitors with improved IL-13 binding kinetics and/or neutralization capacity and/or IL-13 species cross-reactivity. The inventors realized that such advantages would provide for improved therapeutic and prophylactic anti-IL-13 drugs and development of these.

### SUMMARY OF THE INVENTION

The invention provides improved anti-IL-13 immunoglobulin single variable domains, antagonists and compositions comprising these, methods and uses.

In one aspect, the present invention provides an anti-interleukin-13 (IL-13) immunoglobulin single variable domain comprising an amino acid sequence that is identical to DOM10-53-474 (SEQ ID NO: 1), with the exception that the amino acid sequence has 1, 2, 3, 4 or 5 amino acid changes compared to DOM10-53-474 (SEQ ID NO: 1) and wherein the single variable domain has a valine at position 28 according to Kabat numbering, optionally wherein the single variable domain does not consist of DOM10-53-616 (SEQ ID NO: 5).

In a second aspect, the present invention provides an anti-interleukin-13 (IL-13) immunoglobulin single variable domain comprising an amino acid sequence that is identical to DOM10-53-474, with the exception that the amino acid sequence has 1, 2, 3, 4 or 5 amino acid changes compared to DOM10-53-474 (SEQ ID NO: 1) and wherein the single variable domain has the sequence XGX′X″, wherein the G is at position 54 according to Kabat numbering and
X=H or K;
X′=G or K;
X″=K or I, and
   optionally wherein the single variable domain does not consist of DOM10-53-616 (SEQ ID NO: 5).

In a further aspect, the invention provides an anti-interleukin-13 (IL-13) immunoglobulin single variable domain, wherein the variable domain is DOM10-53-546 (SEQ ID NO: 2); DOM10-53-567 (SEQ ID NO: 3); DOM10-53-568 (SEQ ID NO: 4); or DOM10-53-616 (SEQ ID NO: 5). In a further aspect, the invention provides an anti-interleukin-13 (IL-13) immunoglobulin single variable domain, wherein the variable domain is DOM10-53-546 (SEQ ID NO: 2); DOM10-53-567 (SEQ ID NO: 3) or DOM10-53-568 (SEQ ID NO: 4).

An aspect of the invention provides anti-interleukin-13 (IL-13) immunoglobulin single variable domain encoded by the nucleotide sequence of DOM10-53-546 (SEQ ID NO: 6); DOM10-53-567 (SEQ ID NO: 7); DOM10-53-568 (SEQ ID NO: 8); or DOM10-53-616 (SEQ ID NO: 9), optionally wherein the single variable domain does not consist of DOM10-53-616 (SEQ ID NO: 5).

An aspect of the invention provides a polypeptide comprising an amino acid sequence that is at least 99% identical, or 100% identical, to DOM10-53-546 (SEQ ID NO: 2); DOM10-53-567 (SEQ ID NO: 3); DOM10-53-568 (SEQ ID NO: 4); or DOM10-53-616 (SEQ ID NO: 5), optionally wherein the polypeptide does not comprise DOM10-53-616 (SEQ ID NO: 5).

The invention also relates to antagonists, fusion proteins and devices comprising such single variable domains, uses, methods and compositions. The antagonists are useful for addressing IL-13-mediated diseases and conditions in patients, such as human patients, for example for treating and/or preventing lung disease such as asthma, COPD or influenza.

Further embodiments of the invention of different scope are contemplated herein, and as disclosed as follows.

### BRIEF DESCRIPTION OF THE FIGURES

FIG. 1: Map of pDOM5 vector.

FIG. 2: (a) Amino acid sequences of anti-IL-13 immunoglobulin single variable domains of the invention as well as prior art anti-IL-13 single variable domain DOM10-53-474 (SEQ ID NO: 1); and (b) nucleotide sequences of the CDRs (according to Kabat) of the single variable domains.

FIG. 3: Nucleotide sequences encoding anti-IL-13 immunoglobulin single variable domains of the invention as well as prior art anti-IL-13 single variable domain DOM10-53-474.

FIG. 4: (a) & (b) Alignment of amino acid sequences of anti-IL-13 immunoglobulin variable domains of the invention versus the amino acid sequence of DOM10-53-474 (SEQ ID NO: 1). Numbering is according to Kabat. Amino acid residue differences versus DOM10-53-474 (SEQ ID NO: 1) are indicated by an amino acid (single letter format) at the positions where differences occur. Where there is no difference in amino acid at a particular position versus DOM10-53-474, this is indicated by “.”. CDRs are underlined.
(c) & (d) Alignment of amino acid sequences of anti-IL-13 immunoglobulin variable domains of the invention versus the amino acid sequence of DOM10-53-474 (SEQ ID NO: 1). Numbering is according to Chothia. Amino acid residue differences versus DOM10-53-474 (SEQ ID NO: 1) are indicated by an amino acid (single letter format) at the positions where differences occur. Where there is no difference in amino acid at a particular position versus DOM10-53-474, this is indicated by “.”. CDRs are underlined.

FIG. 5: Trypsin digests of anti-IL-13 immunoglobulin single variable domains of the invention as well as prior art anti-IL-13 single variable domain DOM10-53-474 (SEQ ID NO: 1).

### DETAILED DESCRIPTION

Within this specification the invention has been described, with reference to embodiments, in a way which enables a clear and concise specification to be written. It is intended and should be appreciated that embodiments may be variously combined or separated without parting from the invention.

Unless defined otherwise, all technical and scientific terms used herein have the same meaning as commonly understood by one of ordinary skill in the art (e.g, in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods.

The term “amino acid changes compared to DOM10-53-474 (SEQ ID NO: 1)” includes within its scope amino acid changes where each change is either an amino acid substitution, deletion or addition. In one embodiment, only amino acid substitutions are meant by the term.

As used herein, the term “ligand” refers to a compound that comprises at least one peptide, polypeptide or protein moiety that has a binding site with binding specificity for IL-13. The ligands according to the invention optionally comprise immunoglobulin variable domains which have different binding specificities, and do not contain variable domain pairs which together form a binding site for target compound (i.e., do not comprise an immunoglobulin heavy chain variable domain and an immunoglobulin light chain variable domain that together form a binding site for IL-13). Optionally, each domain which has a binding site that has binding specificity for a target is an immunoglobulin single variable domain (e.g, immunoglobulin single heavy chain variable domain (e.g, V_{H}, V_{HH}), immunoglobulin single light chain variable domain (e.g, V_{L})) that has binding specificity for a desired target (e.g, IL-13). Each polypeptide domain which has a binding site that has binding specificity for a target (e.g, IL-13) can also comprise one or more complementarity determining regions (CDRs) of an antibody or antibody fragment (e.g, an immunoglobulin single variable domain) that has binding specificity for a desired target (e.g, IL-13) in a suitable format, such that the binding domain has binding specificity for the target. For example, the CDRs can be grafted onto a suitable protein scaffold or skeleton, such as an affibody, a SpA scaffold, an LDL receptor class A domain, or an EGF domain. Further, the ligand can be bivalent (heterobivalent) or multivalent (heteromultivalent) as described herein. Thus, “ligands” include polypeptides that comprise two dAbs wherein each dAb binds to a different target (e.g, IL-4, IL-13). Ligands also include polypeptides that comprise at least two dAbs that bind different targets (or the CDRs of dAbs) in a suitable format, such as an antibody format (e.g, IgG-like format, scFv, Fab, Fab′, F(ab′)₂) or a suitable protein scaffold or skeleton, such as an affibody, a SpA scaffold, an LDL receptor class A domain, an EGF domain, avimer and dual- and multi-specific ligands as described herein.

The polypeptide domain which has a binding site that has binding specificity for a target (e.g, IL-13) can also be a protein domain comprising a binding site for a desired target, e.g, a protein domain is selected from an affibody, a SpA domain, an LDL receptor class A domain, an avimer (see, e.g, U.S. Patent Application Publication Nos. 2005/0053973, 2005/0089932, 2005/0164301). If desired, a “ligand” can further comprise one or more additional moieties that can each independently be a peptide, polypeptide or protein moiety or a non-peptidic moiety (e.g, a polyalkylene glycol, a lipid, a carbohydrate). For example, the ligand can further comprise a half-life extending moiety as described herein (e.g, a polyalkylene glycol moiety, a moiety comprising albumin, an albumin fragment or albumin variant, a moiety comprising transferrin, a transferrin fragment or transferrin variant, a moiety that binds albumin, a moiety that binds neonatal Fc receptor).

“Dual-specific ligand” refers to a ligand comprising a first antigen or epitope binding site (e.g., first immunoglobulin single variable domain) and a second antigen or epitope binding site (e.g., second immunoglobulin single variable domain), wherein the binding sites or variable domains are capable of binding to two antigens (e.g., different antigens or two copies of the same antigen) or two epitopes on the same antigen which are not normally bound by a monospecific immunoglobulin. For example, the two epitopes may be on the same antigen, but are not the same epitope or sufficiently adjacent to be bound by a monospecific ligand. In one embodiment, dual specific ligands according to the invention are composed of binding sites or variable domains which have different specificities, and do not contain mutually complementary variable domain pairs (ie VH/VL pairs) which have the same specificity (ie, do not form a unitary binding site).

As used herein, the phrase “target” refers to a biological molecule (e.g, peptide, polypeptide, protein, lipid, carbohydrate) to which a polypeptide domain which has a binding site can bind. The target can be, for example, an intracellular target (e.g, an intracellular protein target), a soluble target (e.g, a secreted protein such as IL-4, IL-13), or a cell surface target (e.g, a membrane protein, a receptor protein). In one embodiment, the target is IL-4 or IL-13.

The phrase “immunoglobulin single variable domain” refers to an antibody variable domain (V_{H}, V_{HH}, V_{L}) that specifically binds an antigen or epitope independently of different or other V regions or domains. An immunoglobulin single variable domain can be present in a format (e.g, homo- or hetero-multimer) with other variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is an “immunoglobulin single variable domain” as the term is used herein. A “single immunoglobulin variable domain” is the same as an “immunoglobulin single variable domain” as the term is used herein. A “single antibody variable domain” or an “antibody single variable domain” is the same as an “immunoglobulin single variable domain” as the term is used herein. An immunoglobulin single variable domain is in one embodiment a human antibody variable domain, but also includes single antibody variable domains from other species such as rodent (for example, as disclosed in WO 00/29004, the contents of which are incorporated herein by reference in their entirety), nurse shark and Camelid V_{HH} dAbs. Camelid V_{HH} are immunoglobulin single variable domain polypeptides that are derived from species including camel, llama, alpaca, dromedary, and guanaco, which produce heavy chain antibodies naturally devoid of light chains. The V_{HH} may be humanized.

The immunoglobulin single variable domains (dAbs) described herein contain complementarity determining regions (CDR1, CDR2 and CDR3). The locations of CDRs and frame work (FR) regions and a numbering system have been defined by Kabat et al. (Kabat, E. A. et al., Sequences of Proteins of Immunological Interest, Fifth Edition, U.S. Department of Health and Human Services, U.S. Government Printing Office (1991)). The amino acid sequences of the CDRs (CDR1, CDR2, CDR3) of the V_{H} and V_{L} (V_{κ}) dAbs disclosed herein will be readily apparent to the person of skill in the art based on the well known Kabat amino acid numbering system and definition of the CDRs. According to the Kabat numbering system heavy chain CDR-H3 have varying lengths, insertions are numbered between residue H100 and H101 with letters up to K (i.e. H100, H100A . . . H100K, H101). CDRs can alternatively be determined using the system of Chothia (Chothia et al., (1989) Conformations of immunoglobulin hypervariable regions; Nature 342, p 877-883), according to AbM or according to the Contact method as follows. See http://www.bioinf.org.uk/abs/ for suitable methods for determining CDRs.

Once each residue has been numbered, one can then apply the following CDR definitions (“-” means same residue numbers as shown for Kabat):

Kabat—most commonly used method based on sequence variability
(using Kabat numbering):
CDR H1: 31-35/35A/35B
CDR H2: 50-65

CDR H3: 95-102
CDR L1: 24-34
CDR L2: 50-56
CDR L3: 89-97
   Chothia—based on location of the structural loop regions
(using Chothia numbering):
CDR H1: 26-32
CDR H2: 52-56
CDR H3: 95-102
CDR L1: 24-34
CDR L2: 50-56
CDR L3: 89-97
   AbM—compromise between Kabat and Chothia

| (using Kabat numbering): | (using Chothia numbering): |
|---|---|
| CDR H1: 26-35/35A/35B | 26-35 |
| CDR H2: 50-58 | — |
| CDR H3: 95-102 | — |
| CDR L1: 24-34 | — |
| CDR L2: 50-56 | — |
| CDR L3: 89-97 | — |

Contact—based on crystal structures and prediction of contact residues with antigen

| (using Kabat numbering): | (using Chothia numbering): |
|---|---|
| CDR H1: 30-35/35A/35B | 30-35 |
| CDR H2: 47-58 | — |
| CDR H3: 93-101 | — |
| CDR L1: 30-36 | — |
| CDR L2: 46-55 | — |
| CDR L3: 89-96 | — |

As used herein “interleukin-4” (IL-4) refers to naturally occurring or endogenous mammalian IL-4 proteins and to proteins having an amino acid sequence which is the same as that of a naturally occurring or endogenous corresponding mammalian IL-4 protein (e.g, recombinant proteins, synthetic proteins (i.e., produced using the methods of synthetic organic chemistry)). Accordingly, as defined herein, the term includes mature IL-4 protein, polymorphic or allelic variants, and other isoforms of an IL-4 and modified or unmodified forms of the foregoing (e.g, lipidated, glycosylated). Naturally occurring or endogenous IL-4 includes wild type proteins such as mature IL-4, polymorphic or allelic variants and other isoforms and mutant forms which occur naturally in mammals (e.g, humans, non-human primates). Such proteins can be recovered or isolated from a source which naturally produces IL-4, for example. These proteins and proteins having the same amino acid sequence as a naturally occurring or endogenous corresponding IL-4, are referred to by the name of the corresponding mammal. For example, where the corresponding mammal is a human, the protein is designated as a human IL-4. Several mutant IL-4 proteins are known in the art, such as those disclosed in WO 03/038041.

As used herein “interleukin-13” (IL-13) refers to naturally occurring or endogenous mammalian IL-13 proteins and to proteins having an amino acid sequence which is the same as that of a naturally occurring or endogenous corresponding mammalian IL-13 protein (e.g, recombinant proteins, synthetic proteins (i.e., produced using the methods of synthetic organic chemistry)). Accordingly, as defined herein, the term includes mature IL-13 protein, polymorphic or allelic variants, and other isoforms of IL-13 (e.g, produced by alternative splicing or other cellular processes), and modified or unmodified forms of the foregoing (e.g, lipidated, glycosylated). Naturally occurring or endogenous IL-13 include wild type proteins such as mature IL-13, polymorphic or allelic variants and other isoforms and mutant forms which occur naturally in mammals (e.g, humans, non-human primates). For example, as used herein IL-13 encompasses the human IL-13 variant in which Arg at position 110 of mature human IL-13 is replaced with Gln (position 110 of mature IL-13 corresponds to position 130 of the precursor protein) which is associated with asthma (atopic and nonatopic asthma) and other variants of IL-13. (Heinzmann et al., Hum Mol. Genet. 9:549-559 (2000).) Such proteins can be recovered or isolated from a source which naturally produces IL-13, for example. These proteins and proteins having the same amino acid sequence as a naturally occurring or endogenous corresponding IL-13, are referred to by the name of the corresponding mammal. For example, where the corresponding mammal is a human, the protein is designated as a human IL-13. Several mutant IL-13 proteins are known in the art, such as those disclosed in WO 03/035847.

“Affinity” and “avidity” are terms of art that describe the strength of a binding interaction. With respect to the ligands of the invention, avidity refers to the overall strength of binding between the targets (e.g, first target and second target) on the cell and the ligand. Avidity is more than the sum of the individual affinities for the individual targets.

As used herein, “toxin moiety” refers to a moiety that comprises a toxin. A toxin is an agent that has deleterious effects on and/or alters cellular physiology (e.g, causes cellular necrosis, apoptosis or inhibits cellular division).

As used herein, the term “dose” refers to the quantity of ligand administered to a subject all at one time (unit dose), or in two or more administrations over a defined time interval. For example, dose can refer to the quantity of ligand (e.g, ligand comprising an immunoglobulin single variable domain that binds IL-13) administered to a subject over the course of one day (24 hours) (daily dose), two days, one week, two weeks, three weeks or one or more months (e.g, by a single administration, or by two or more administrations). The interval between doses can be any desired amount of time.

As used herein “complementary” refers to when two immunoglobulin domains belong to families of structures which form cognate pairs or groups or are derived from such families and retain this feature. For example, a V_{H} domain and a V_{L} domain of an antibody are complementary; two V_{H} domains are not complementary, and two V_{L} domains are not complementary. Complementary domains may be found in other members of the immunoglobulin superfamily, such as the V_{α} and V_{β} (or γ and δ) domains of the T-cell receptor. Domains which are artificial, such as domains based on protein scaffolds which do not bind epitopes unless engineered to do so, are non-complementary. Likewise, two domains based on (for example) an immunoglobulin domain and a fibronectin domain are not complementary.

As used herein, “immunoglobulin” refers to a family of polypeptides which retain the immunoglobulin fold characteristic of antibody molecules, which contain two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signaling (for example, receptor molecules, such as the PDGF receptor). The present invention is applicable to all immunoglobulin superfamily molecules which possess binding domains. In one embodiment, the present invention relates to antibodies.

As used herein “domain” refers to a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. By single antibody variable domain is meant a folded polypeptide domain comprising sequences characteristic of antibody variable domains. It therefore includes complete antibody variable domains and modified variable domains, for example in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain. Thus, each ligand comprises at least two different domains.

The term “library” refers to a mixture of heterogeneous polypeptides or nucleic acids. The library is composed of members, each of which has a single polypeptide or nucleic acid sequence. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Optionally, each individual organism or cell contains only one or a limited number of library members. Advantageously, the nucleic acids are incorporated into expression vectors, in order to allow expression of the polypeptides encoded by the nucleic acids. In one aspect, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

As used herein an “antibody” refers to IgG, IgM, IgA, IgD or IgE or a fragment (such as a Fab, F(ab′)₂, Fv, disulphide linked Fv, scFv, closed conformation multispecific antibody, disulphide-linked scFv, diabody) whether derived from any species naturally producing an antibody, or created by recombinant DNA technology; whether isolated from, for example, serum, B-cells, hybridomas, transfectomas, yeast or bacteria.

As described herein an “antigen” is a molecule that is bound by a binding domain according to the present invention. Typically, antigens are bound by antibody ligands and are capable of raising an antibody response in vivo. It may be, for example, a polypeptide, protein, nucleic acid or other molecule. Generally, the dual-specific ligands according to the invention are selected for target specificity against two particular targets (e.g, antigens). In the case of conventional antibodies and fragments thereof, the antibody binding site defined by the variable loops (L1, L2, L3 and H1, H2, H3) is capable of binding to the antigen.

An “epitope” is a unit of structure conventionally bound by an immunoglobulin V_{H}/V_{L} pair. Epitopes define the minimum binding site for an antibody, and thus represent the target of specificity of an antibody. In the case of a single domain antibody, an epitope represents the unit of structure bound by a variable domain in isolation.

“Universal framework” refers to a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. The invention provides for the use of a single framework, or a set of such frameworks, which has been found to permit the derivation of virtually any binding specificity through variation in the hypervariable regions alone.

The phrase, “half-life,” refers to the time taken for the serum concentration of the ligand to reduce by 50%, in vivo, for example due to degradation of the ligand and/or clearance or sequestration of the dual-specific ligand by natural mechanisms. The ligands of the invention are stabilized in vivo and their half-life increased by binding to molecules which resist degradation and/or clearance or sequestration. Typically, such molecules are naturally occurring proteins which themselves have a long half-life in vivo. The half-life of a ligand is increased if its functional activity persists, in vivo, for a longer period than a similar ligand which is not specific for the half-life increasing molecule. Thus a ligand specific for HSA and two target molecules is compared with the same ligand wherein the specificity to HSA is not present, that is does not bind HSA but binds another molecule. For example, it may bind a third target on the cell. Typically, the half-life is increased by 10%, 20%, 30%, 40%, 50% or more. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half life are possible.

As used herein, the terms “low stringency,” “medium stringency,” “high stringency,” or “very high stringency” conditions describe conditions for nucleic acid hybridization and washing. Guidance for performing hybridization reactions can be found in Current Protocols in Molecular Biology, John Wiley & Sons, N.Y. (1989), 6.3.1-6.3.6, which is incorporated herein by reference in its entirety. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and optionally (4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

Sequences similar or homologous (e.g, at least about 70% sequence identity) to the sequences disclosed herein are also part of the invention. In some embodiments, the sequence identity at the amino acid level can be about 80%, 85%, 90%, 91%, 92%, 93%, 94%, 95%, 96%, 97%, 98%, 99% or higher. At the nucleic acid level, the sequence identity can be about 70%, 75%, 80%, 85%, 90%, 91%, 92%, 93%, 94%, 95%, 96%, 97%, 98%, 99% or higher. Alternatively, substantial identity exists when the nucleic acid segments will hybridize under selective hybridization conditions (e.g, very high stringency hybridization conditions), to the complement of the strand. The nucleic acids may be present in whole cells, in a cell lysate, or in a partially purified or substantially pure form.

Calculations of “homology” or “sequence identity” or “similarity” between two sequences (the terms are used interchangeably herein) are performed as follows. The sequences are aligned for optimal comparison purposes (e.g, gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least about 30%, optionally at least about 40%, optionally at least about 50%, optionally at least about 60%, and optionally at least about 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “homology” is equivalent to amino acid or nucleic acid “identity”). The percent identity between the two sequences is a function of the number of identical positions shared by the sequences, taking into account the number of gaps, and the length of each gap, which need to be introduced for optimal alignment of the two sequences.

Amino acid and nucleotide sequence alignments and homology, similarity or identity, as defined herein are optionally prepared and determined using the algorithm BLAST 2 Sequences, using default parameters (Tatusova, T. A. et al., FEMS Microbiol Lett, 174:187-188 (1999)). Alternatively, the BLAST algorithm (version 2.0) is employed for sequence alignment, with parameters set to default values. BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul, 1990, Proc. Natl. Acad. Sci. USA 87(6):2264-8.

Unless defined otherwise, all technical and scientific terms used herein have the same meaning as commonly understood by one of ordinary skill in the art (e.g, in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

As used herein, the term “antagonist of IL-13” or “anti-IL-13 antagonist” or the like refers to an agent (e.g, a molecule, a compound) which binds IL-13 and can inhibit a (i.e., one or more) function of IL-13. For example, an antagonist of IL-13 can inhibit the binding of IL-13 to a receptor for IL-13 and/or inhibit signal transduction mediated through a receptor for IL-13. Accordingly, IL-13-mediated processes and cellular responses can be inhibited with an antagonist of IL-13.

As used herein, “peptide” refers to about two to about 50 amino acids that are joined together via peptide bonds.

As used herein, “polypeptide” refers to at least about 50 amino acids that are joined together by peptide bonds. Polypeptides generally comprise tertiary structure and fold into functional domains.

As used herein, a peptide or polypeptide (e.g, a domain antibody (dAb)) that is “resistant to protease degradation” is not substantially degraded by a protease when incubated with the protease under conditions suitable for protease activity. A polypeptide (e.g, a dAb) is not substantially degraded when no more than about 25%, no more than about 20%, no more than about 15%, no more than about 14%, no more than about 13%, no more than about 12%, no more than about 11%, no more than about 10%, no more than about 9%, no more than about 8%, no more than about 7%, no more than about 6%, no more than about 5%, no more than about 4%, no more than about 3%, no more that about 2%, no more than about 1%, or substantially none of the protein is degraded by protease after incubation with the protease for about one hour at a temperature suitable for protease activity. For example at 37 or 50 degrees C. Protein degradation can be assessed using any suitable method, for example, by SDS-PAGE or by functional assay (e.g, ligand binding) as described herein.

As used herein, “display system” refers to a system in which a collection of polypeptides or peptides are accessible for selection based upon a desired characteristic, such as a physical, chemical or functional characteristic. The display system can be a suitable repertoire of polypeptides or peptides (e.g, in a solution, immobilized on a suitable support). The display system can also be a system that employs a cellular expression system (e.g, expression of a library of nucleic acids in, e.g, transformed, infected, transfected or transduced cells and display of the encoded polypeptides on the surface of the cells) or an acellular expression system (e.g, emulsion compartmentalization and display). Exemplary display systems link the coding function of a nucleic acid and physical, chemical and/or functional characteristics of a polypeptide or peptide encoded by the nucleic acid. When such a display system is employed, polypeptides or peptides that have a desired physical, chemical and/or functional characteristic can be selected and a nucleic acid encoding the selected polypeptide or peptide can be readily isolated or recovered. A number of display systems that link the coding function of a nucleic acid and physical, chemical and/or functional characteristics of a polypeptide or peptide are known in the art, for example, bacteriophage display (phage display, for example phagemid display), ribosome display, emulsion compartmentalization and display, yeast display, puromycin display, bacterial display, display on plasmid, covalent display and the like. (See, e.g, EP 0436597 (Dyax), U.S. Pat. No. 6,172,197 (McCafferty et al.), U.S. Pat. No. 6,489,103 (Griffiths et al.))

As used herein, “repertoire” refers to a collection of polypeptides or peptides that are characterized by amino acid sequence diversity. The individual members of a repertoire can have common features, such as common structural features (e.g, a common core structure) and/or common functional features (e.g, capacity to bind a common ligand (e.g, a generic ligand or a target ligand, IL-13)).

As used herein, “functional” describes a polypeptide or peptide that has biological activity, such as specific binding activity. For example, the term “functional polypeptide” includes an antibody or antigen-binding fragment thereof that binds a target antigen through its antigen-binding site.

As used herein, “generic ligand” refers to a ligand that binds a substantial portion (e.g, substantially all) of the functional members of a given repertoire. A generic ligand (e.g, a common generic ligand) can bind many members of a given repertoire even though the members may not have binding specificity for a common target ligand. In general, the presence of a functional generic ligand-binding site on a polypeptide (as indicated by the ability to bind a generic ligand) indicates that the polypeptide is correctly folded and functional. Suitable examples of generic ligands include superantigens, antibodies that bind an epitope expressed on a substantial portion of functional members of a repertoire, and the like.

“Superantigen” is a term of art that refers to generic ligands that interact with members of the immunoglobulin superfamily at a site that is distinct from the target ligand-binding sites of these proteins. Staphylococcal enterotoxins are examples of superantigens which interact with T-cell receptors. Superantigens that bind antibodies include Protein G, which binds the IgG constant region (Bjorck and Kronvall, J. Immunol., 133:969 (1984)); Protein A which binds the IgG constant region and V_{H} domains (Forsgren and Sjoquist, J. Immunol., 97:822 (1966)); and Protein L which binds V_{L} domains (Bjorck, J. Immunol., 140:1194 (1988)).

As used herein, “antibody format” refers to any suitable polypeptide structure in which one or more antibody variable domains can be incorporated so as to confer binding specificity for antigen on the structure. A variety of suitable antibody formats are known in the art, such as, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g, a Fv fragment (e.g, single chain Fv (scFv), a disulfide bonded Fv), a Fab fragment, a Fab′ fragment, a F(ab′)₂ fragment), a single antibody variable domain (e.g, a dAb, V_{H}, V_{HH}, V_{L}), and modified versions of any of the foregoing (e.g, modified by the covalent attachment of polyethylene glycol or other suitable polymer or a humanized V_{HH}).

As used herein, “hydrodynamic size” refers to the apparent size of a molecule (e.g, a protein molecule, ligand) based on the diffusion of the molecule through an aqueous solution. The diffusion or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation of the protein.

As referred to herein, the term “competes” means that the binding of a first target (e.g., IL-13) to its cognate target binding domain (e.g., immunoglobulin single variable domain) is inhibited in the presence of a second binding domain (e.g., immunoglobulin single variable domain) that is specific for said cognate target. For example, binding may be inhibited sterically, for example by physical blocking of a binding domain or by alteration of the structure or environment of a binding domain such that its affinity or avidity for a target is reduced. See WO2006038027 for details of how to perform competition ELISA and competition BiaCore experiments to determine competition between first and second binding domains, the details of which are incorporated herein by reference to provide explicit disclosure for use in the present invention.

The present inventors realized that mutation of position 28 (Kabat numbering) in DOM10-53-474 (SEQ ID NO: 1) provides dAb derivatives that are much more potent for IL-13 binding. Additional advantages may also be produced, such as cross reactivity between human and at least one non-human primate IL-13 (eg cyno and/or rhesus). Furthermore, good expression in prokaryotic cells may also be produced.

In one aspect, therefore, the present invention provides an anti-interleukin-13 (IL-13) immunoglobulin single variable domain comprising an amino acid sequence that is identical to DOM10-53-474 (SEQ ID NO: 1), with the exception that the amino acid sequence has 1, 2, 3, 4 or 5 amino acid changes compared to DOM10-53-474 (SEQ ID NO: 1) and wherein the single variable domain has a valine at position 28 according to Kabat numbering optionally wherein the single variable domain does not consist of DOM10-53-616 (SEQ ID NO: 5).

We also refer herein to the term “dAb” (domain antibody). A dAb is an “immunoglobulin single variable domain”.

The inventors also realized that DOM10-53-474 dAb derivatives having the sequence motif XGX′X″ (wherein the G is at position 54 according to Kabat numbering and X=H or K; X′=G or K; and X″=K or I) are much more potent for IL-13 binding. Additional advantages may also be produced, such as cross reactivity between human and at least one non-human primate IL-13 (e.g, cyno and/or rhesus). Furthermore, the advantage of increased expression in prokaryotic cells may also be produced.

In a second aspect, therefore, the present invention provides an anti-interleukin-13 (IL-13) immunoglobulin single variable domain comprising an amino acid sequence that is identical to DOM10-53-474, with the exception that the amino acid sequence has 1, 2, 3, 4 or 5 amino acid changes compared to DOM10-53-474 (SEQ ID NO: 1) and wherein the single variable domain has the sequence XGX′X″, wherein the G is at position 54 according to Kabat numbering and
X=H or K;
X′=G or K;
X″=K or I, and
   optionally wherein the single variable domain does not consist of DOM10-53-616 (SEQ ID NO: 5).

In one embodiment of the second aspect, the variable domain has a valine at position 28 according to Kabat numbering.

In one embodiment of the second aspect, the single variable domain comprises:—
KGGK;
XGKI; or
HGKI; wherein G (or the first G for KGGK) is position 54 according to Kabat numbering.

In one embodiment of the second aspect, CDR2 (according to Kabat) of the single variable domain has the sequence SIDW[Z]TYYADSVKG, wherein [Z] is selected from XGX′X″, KGGK; XGKI and HGKI as defined above.

In either aspect, the variable domain can optionally have an amino acid change (versus DOM10-53-474 (SEQ ID NO: 1)) at one or more of position 30, 53, 55 and 56 (according to Kabat numbering). The variable domain optionally has

a) proline at position 30 (according to Kabat numbering), and/or
b) lysine at position 53 (according to Kabat numbering), and/or
c) glycine or lysine at position 55 (according to Kabat numbering), and/or
d) isoleucine or lysine at position 56 (according to Kabat numbering).

In one embodiment, the single variable domain has lysine at position 55 and isoleucine at position 56 (according to Kabat numbering).

In one aspect, the invention provides an anti-interleukin-13 (IL-13) immunoglobulin single variable domain, wherein the CDRs (eg as determined by Kabat) are identical to the CDRs of DOM10-53-474 (SEQ ID NO: 1) and wherein the single variable domain comprises comprising valine at position 28 according to Kabat numbering. Optionally, the amino acid sequence of the single variable domain has 1, 2, 3, 4 or 5 amino acid changes compared to DOM10-53-474 (SEQ ID NO: 1), wherein one or more changes are optionally in a CDR, eg CDR2 according to Kabat or Chothia. Optionally the single variable domain does not consist of DOM10-53-616 (SEQ ID NO: 5).

In one aspect, the invention provides an anti-interleukin-13 (IL-13) immunoglobulin single variable domain, wherein the CDRs (eg as determined by Kabat) are identical to the CDRs of DOM10-53-474 (SEQ ID NO: 1) with the exception that the single variable domain comprises comprising the SIDW[Z]TYYADSVKG, XGX′X″, KGGK; XGKI or HGKI motif as defined above. Optionally, the single variable domain has valine at position 28 according to Kabat numbering. Optionally the single variable domain does not consist of DOM10-53-616 (SEQ ID NO: 5).

In a further aspect, the invention provides an anti-interleukin-13 (IL-13) immunoglobulin single variable domain, wherein the variable domain is DOM10-53-546 (SEQ ID NO: 2); DOM10-53-567 (SEQ ID NO: 3); DOM10-53-568 (SEQ ID NO: 4); or DOM10-53-616 (SEQ ID NO: 5). In a further aspect, the invention provides an anti-interleukin-13 (IL-13) immunoglobulin single variable domain, wherein the variable domain is DOM10-53-546 (SEQ ID NO: 2); DOM10-53-567 (SEQ ID NO: 3) or DOM10-53-568 (SEQ ID NO: 4).

An aspect of the invention provides an anti-interleukin-13 (IL-13) immunoglobulin single variable domain encoded by the nucleotide sequence of DOM10-53-546 (SEQ ID NO: 6); DOM10-53-567 (SEQ ID NO: 7); DOM10-53-568 (SEQ ID NO: 8); or DOM10-53-616 (SEQ ID NO: 9). Optionally the single variable domain does not consist of DOM10-53-616 (SEQ ID NO: 5).

An aspect of the invention provides anti-interleukin-13 (IL-13) immunoglobulin single variable domain that specifically binds to human IL-13 and at least one non-human primate IL-13. For example, the variable domain specifically binds human IL-13 and Cynomolgus monkey and/or rhesus IL-13 and/or baboon IL-13, e.g., human and Cynomolgus monkey IL-13 or human and rhesus IL-13 or human and baboon IL-13 or human, rhesus and Cynomolgus monkey IL-13. The single variable domain may be optionally according to any preceding aspect of the invention. Optionally the single variable domain does not consist of DOM10-53-616 (SEQ ID NO: 5). In one embodiment, the variable domain binds human IL-13 and the or each non-human primate IL-13 with a dissociation constant (Kd) of about 5 nM or less, optionally about 4 nM or less, about 3 nM or less or about 2 nM or less or about 1 nM or less. In one embodiment, the variable domain binds (i) human IL-13 with a dissociation constant (Kd) of about 1 nM or less, optionally about 500 pM or less, optionally about 250 pM or less, optionally about 150 pM or less, optionally about 100 pM or less, optionally about 1 nM to about 10 pM, about 1 nM to about 50 pM or about 1 nM to about 70 pM, optionally about 500 pM to about 10 pM, about 500 pM to about 50 pM or about 500 pM to about 70 pM, optionally 250 pM to about 10 pM, about 250 pM to about 50 pM or about 250 pM to about 70 pM, optionally about 150 pM to about 10 pM, about 150 pM to about 50 pM or about 150 pM to about 70 pM, or optionally about 100 pM to about 10 pM, about 100 pM to about 50 pM or about 100 pM to about 70 pM; and (ii) the non-human (e.g., cynomolgus monkey, rhesus or baboon) IL-13 with a dissociation constant (Kd) of 5 nM or less, optionally 4, 3, 2 or 1 n M or less, optionally 5 to 1 nM. In one embodiment, additionally or alternatively, the single variable domain comprises a valine at position 28 according to Kabat numbering. In one embodiment, additionally or alternatively, the single variable domain comprises the sequence XGX′X″, wherein the G is at position 54 according to Kabat numbering and
X=H or K;
X′=G or K;
X″=K or I.

In one embodiment, additionally or alternatively, the variable domain has

a) proline at position 30 (according to Kabat numbering), and/or
b) lysine at position 53 (according to Kabat numbering), and/or
c) glycine or lysine at position 55 (according to Kabat numbering), and/or
d) isoleucine or lysine at position 56 (according to Kabat numbering).

In one embodiment, additionally or alternatively, the variable domain has lysine at position 55 and isoleucine at position 56 (according to Kabat numbering).

Single variable domains of the invention, in all aspects, are advantageous because they display one or more of the following advantages:—
(i) Good potency against human IL-13 (better potency than DOM10-53-474);
(ii) Good potency against Cynomolgus monkey IL-13 (better potency than DOM10-53-474);
(iii) Good potency against rhesus IL-13 (better potency than DOM10-53-474);
(iv) Good potency against human and a non-human primate (e.g., Cynomolgus monkey, rhesus or baboon) IL-13 (better potency than DOM10-53-474);
(v) Good potency against human, Cynomolgus monkey and rhesus IL-13 (better potency than DOM10-53-474);
(vi) Good expression in prokaryotic cells (optionally better than DOM10-53-474)
(vii) Good neutralization of human IL-13 (better than DOM10-53-474);
(viii) Good neutralization of Cynomolgus monkey IL-13 (better than DOM10-53-474);
(ix) Good neutralization of rhesus IL-13 (better than DOM10-53-474);
(x) Good neutralization of human and a non-human primate (e.g., Cynomolgus monkey, rhesus or baboon) IL-13 (better than DOM10-53-474);
(xi) Good neutralization of human, Cynomolgus monkey and rhesus IL-13 (better than DOM10-53-474);
(xii) Cross-reactivity between more than one species of primate IL-13 (optionally, human and Cynomolgus monkey and/or rhesus IL-13, eg human and Cynomolgus monkey IL-13; or human and rhesus IL-13; or human and baboon IL-13); and
(xiii) Protease stability (optionally, trypsin stability).

Advantages (i) to (v) can be determined, in one embodiment, by surface plasmon resonance, eg by Biacore™. In an embodiment, better potency is indicated by better dissociation constant (Kd).

Advantage (vi) can be determined, in one embodiment, by expression in E. coli. In an embodiment, a single variable domain of the invention expresses well (at least 3 mg/litre, eg at least 5, 10, 15, 20 mg/L in E. Coli.

In an embodiment, a single variable domain of the invention expresses well in Pichia pastoris or Saccharomyces.

Advantages (vii) to (xi) can be determined, in one embodiment, by neutralization determined by ELISA or a standard HEK STAT assay. In an embodiment, neutralization is indicated by EC₅₀.

Advantage (xii) can be determined, in one embodiment, by ELISA, Biacore™ or a standard HEK STAT assay. In an embodiment, cross-reactivity is indicated by dissociation constants (Kd).

Advantage (xiii) can be determined, in one embodiment, as follows: —
0.3 mg/ml single variable domain is mixed with trypsin (e.g., trypsin activity >5000 units/mg) at a ratio of 25:1 dAb:trypsin in PBS buffer. After incubation at 30 degrees centigrade for 24 hours, the presence of active dAb remaining after incubation is determined.

In one embodiment, the presence of active dAb is determines as the percentage dAb activity remaining, and this is determined by surface plasmon resonance (e.g., Biacore™). A percentage activity of at least 20% (e.g., at least 30%, 40% or 50%) after 24 hours is indicative of a protease-stable dAb. In another embodiment, the presence of a protease-stable dAb is determined using ELISA, wherein a protease-stable variable domain has an OD₄₅₀ reading in ELISA of at least 0.404 following incubation. In another embodiment, the presence of active dAb is determined if dAb specifically binds protein A or protein L following incubation. In another embodiment, the presence of active dAb is determined by gel electrophoresis, a protease-stable variable domain displaying substantially a single band in gel electrophoresis following incubation.

In one embodiment, a single variable domain of the invention has advantages (iv) and (x). In another embodiment, a single variable domain of the invention has advantages (v) and (xi). Optionally the single variable domain does not consist of DOM10-53-616 (SEQ ID NO 5).

In one embodiment, the invention provides a single variable domain according to the invention for any use, any advantage, any treatment and/or prophylaxis of any disease or condition disclosed herein. In one embodiment, the invention provides the use of a single variable domain according to the invention in the manufacture of an IL-13 antagonist for any use, any advantage, any treatment and/or prophylaxis of any disease or condition disclosed herein. These statements provide explicit basis for importation into claims herein.

Single variable domains of the present invention (DOM10-53-546, DOM10-53-567, DOM10-53-568 and DOM10-53-616) show good potency against both human and Cynomolgus (cyno) monkey IL-13 as indicated by their dissociation constants (Kd) for IL-13 binding as determined by surface plasmon resonance (e.g., Biacore™). The single variable domains of the present invention are much more potent than DOM10-53-474. DOM10-53-567 and DOM10-53-568 have the best potency for both human and cyno IL-13.

Single variable domains of the present invention (DOM10-53-546, DOM10-53-567, DOM10-53-568 and DOM10-53-616) show cross-reactivity between more than one species of primate IL-13. In one aspect, the invention provides the single variable domains for providing a single variable domain that is cross-reactive between more than one species of primate IL-13 optionally between human and a non-human primate IL-13, optionally between (i) human and Cynomolgus monkey IL-13 species, (ii) human and rhesus IL-13 species, (iii) human, Cynomolgus monkey and rhesus IL-13 species, or (iv) human and baboon IL-13 species. In one aspect, the invention provides the use of a single variable domain of the invention in the manufacture of an IL-13 antagonist that is cross-reactive between more than one species of primate IL-13 optionally between human and a non-human primate IL-13, optionally between (i) human and Cynomolgus monkey IL-13 species, (ii) human and rhesus IL-13 species, (iii) human, Cynomolgus monkey and rhesus IL-13 species, or (iv) human and baboon IL-13 species. The variable domains specifically bind human and a non-human primate IL-13 (in the examples, human, Cynomolgus monkey and rhesus IL-13 species are bound by the variable domains). This is particularly useful, since drug development typically requires testing of lead drug candidates in non-human primate systems such as Cynomolgus monkey and rhesus before the drug is tested in humans. The provision of a drug that can bind human and other primate IL-13 species allows one to test results in these system and make side-by-side comparisons of data using the same drug. This avoids the complication of needing to find a drug that works against a non-human IL-13 and a separate drug that works against human IL-13, and also avoids the need to compare results in humans and non-human primates using non-identical drugs.

Optionally, the binding affinity of the immunoglobulin single variable domain for at least one non-human primate (e.g., cyno and/or rhesus and/or baboon) IL-13 and the binding affinity for human IL-13 differ by no more than a factor of 10, 50, 100, 500 or 1000. In one aspect the invention provides a single variable domain according to the invention for providing affinity of the immunoglobulin single variable domain for at least one non-human primate (e.g., cyno and/or rhesus and/or baboon) IL-13 and the binding affinity for human IL-13 differ by no more than a factor of 10, 50, 100, 500 or 1000. In one aspect the invention provides the use of a single variable domain according to the invention for the manufacture of an IL-13 antagonist, wherein the affinity of the immunoglobulin single variable domain for at least one non-human primate (e.g., cyno and/or rhesus and/or baboon) IL-13 and the binding affinity for human IL-13 differ by no more than a factor of 10, 50, 100, 500 or 1000.

Single variable domains of the present invention (DOM10-53-546, DOM10-53-567, DOM10-53-568 and DOM10-53-616) can neutralize more than one species of primate IL-13. In one aspect, the invention provides a single variable domain according to the invention for neutralizing more than one species of primate IL-13, optionally for neutralizing (i) human and Cynomolgus monkey IL-13 species, (ii) human and rhesus IL-13 species, (iii) human, Cynomolgus monkey and rhesus IL-13 species, or (iv) human and baboon IL-13 species. In one aspect, the invention provides the use of a single variable domain according to the invention for the manufacture of an IL-13 antagonist, for neutralizing more than one species of primate IL-13, optionally for neutralizing (i) human and Cynomolgus monkey IL-13 species, (ii) human and rhesus IL-13 species, (iii) human, Cynomolgus monkey and rhesus IL-13 species, or (iv) human and baboon IL-13 species. As shown by the examples below, DOM10-53-546, DOM10-53-567, DOM10-53-568 and DOM10-53-616 showed neutralization of all forms of IL-13 tested (human, Cynomolgus monkey and rhesus IL-13) in ELISA or HEK STAT assays. DOM10-53-546, DOM10-53-567 and DOM10-53-568 showed much more neutralizing potency for human, Cynomolgus monkey and rhesus IL-13 than DOM10-53-474. DOM10-53-616 was a much more potent neutralizer than DOM10-53-474 for Cynomolgus monkey and rhesus IL-13 and was broadly comparable against human IL-13.

Single variable domains (DOM10-53-567 and DOM10-53-568) are particularly protease stable. In one aspect, the invention provides a single variable domain of the invention for providing a protease stable single variable domain or IL-13 antagonist. In one aspect, the invention provides the use of a single variable domain of the invention in the manufacture of an IL-13 antagonist for providing a protease stable IL-13 antagonist. Protease stability is determined as follows: —
0.3 mg/ml single variable domain is mixed with trypsin (e.g., trypsin activity >5000 units/mg) at a ratio of 25:1 dAb:trypsin in PBS buffer. After incubation at 30 degrees centigrade for 24 hours, the percentage of active dAb remaining after incubation is determined (e.g., using Biacore™). A percentage activity of at least 20% (e.g., at least 30%, 40% or 50%) after 24 hours is indicative of a protease-stable dAb.

Reference is made to WO2008149143. The entire disclosure of this application is incorporated herein by reference, in order to provide further disclosure herein of protease-resistant polypeptides and dAbs; methods for selecting these (including disclosure of different proteases that can be used and selection conditions); uses of these; compositions, ligands and products comprising these; and advantages of such polypeptides and dAbs. These disclosures are explicitly included, and form part of, the present disclosure for use with the present invention.

Polypeptides and peptides have become increasingly important agents in a variety of applications, including industrial applications and use as medical, therapeutic and diagnostic agents. However, in certain physiological states, such as inflammatory states (e.g, COPD) and cancer, the amount of proteases present in a tissue, organ or animal (e.g, in the lung, in or adjacent to a tumor) can increase. This increase in proteases can result in accelerated degradation and inactivation of endogenous proteins and of therapeutic peptides, polypeptides and proteins that are administered to treat disease. Accordingly, some agents that have potential for in vivo use (e.g, use in treating, diagnosing or preventing disease in mammals such as humans) have only limited efficacy because they are rapidly degraded and inactivated by proteases.

Protease resistant polypeptides provide several advantages. For example, protease resistant polypeptides remaining active in vivo longer than protease sensitive agents and, accordingly, remaining functional for a period of time that is sufficient to produce biological effects. A need exists for improved methods to select polypeptides that are resistant to protease degradation and also have desirable biological activity. It would be particularly useful to provide peptides and polypeptides that are resistant to proteases found in fluids and tissues of the gastrointestinal tract (GI tract) and/or pulmonary system, which system includes the lung. Both the GI tract and the pulmonary system are sites for disease and adverse conditions in mammals, eg humans; protease resistant peptide and polypeptide drugs would be advantageous in this context.

Single variable domains of the present invention (DOM10-53-546 and DOM10-53-616) showed much better expression levels than DOM10-53-474 in prokaryotic cells. In one aspect, the invention provides a single variable domain of the invention (eg, DOM10-53-546 and DOM10-53-616) for providing a single variable domain that has expression in prokaryotic cells that is better than expression of DOM10-53-474. In one aspect, the invention provides the use of a single variable domain of the invention (eg, DOM10-53-546 and DOM10-53-616) in the manufacture of an IL-13 antagonist, wherein the single variable domain has expression in prokaryotic cells that is better than expression of DOM10-53-474.

In an embodiment of any preceding aspect of the invention, the single variable domain specifically binds human, cynomolgus monkey and rhesus IL-13. Specific binding is indicated by a dissociation constant Kd of 10 micromolar or less, optionally 1 micromolar or less. Specific binding of an antigen-binding protein to an antigen or epitope can be determined by a suitable assay, including, for example, Scatchard analysis and/or competitive binding assays, such as radioimmunoassays (RIA), enzyme immunoassays such as ELISA and sandwich competition assays, and the different variants thereof.

Binding affinity is optionally determined using surface plasmon resonance (SPR) and Biacore (Karlsson et al., 1991), using a Biacore system (Uppsala, Sweden). The Biacore system uses surface plasmon resonance (SPR, Welford K. 1991, Opt. Quant. Elect. 23:1; Morton and Myszka, 1998, Methods in Enzymology 295: 268) to monitor biomolecular interactions in real time, and uses surface plasmon resonance which can detect changes in the resonance angle of light at the surface of a thin gold film on a glass support as a result of changes in the refractive index of the surface up to 300 nm away. Biacore analysis conveniently generates association rate constants, dissociation rate constants, equilibrium dissociation constants, and affinity constants. Binding affinity is obtained by assessing the association and dissociation rate constants using a Biacore surface plasmon resonance system (Biacore, Inc.). A biosensor chip is activated for covalent coupling of the target according to the manufacturer's (Biacore) instructions. The target is then diluted and injected over the chip to obtain a signal in response units of immobilized material. Since the signal in resonance units (RU) is proportional to the mass of immobilized material, this represents a range of immobilized target densities on the matrix. Dissociation data are fit to a one-site model to obtain k_{off}+/−s.d. (standard deviation of measurements). Pseudo-first order rate constant (Kd's) are calculated for each association curve, and plotted as a function of protein concentration to obtain kₒₙ+/−s.e. (standard error of fit). Equilibrium dissociation constants for binding, Kd's, are calculated from SPR measurements as k_{off}/kₒₙ.

In an embodiment of any preceding aspect of the invention, the variable domain neutralises human IL-13 in a standard HEK STAT assay with an EC₅₀, of about 0.1 to about 2.0 nM, optionally about 0.2 to about 2.0 nM, about 0.3 to about 1.5 nM, about 0.2 to about 1.0 nM or about 0.3 to about 1.0 nM. In one aspect, the invention provides a single variable domain of the invention for neutralising human IL-13 in a standard HEK STAT assay with an EC₅₀ of about 0.1 to about 2.0 nM, optionally about 0.2 to about 2.0 nM, about 0.3 to about 1.5 nM, about 0.2 to about 1.0 nM or about 0.3 to about 1.0 nM. In one aspect, the invention provides the use of a single variable domain of the invention in the manufacture of an IL-13 antagonist, wherein the variable domain or antagonist neutralises human IL-13 in a standard HEK STAT assay with an EC₅₀ of about 0.1 to about 2.0 nM, optionally about 0.2 to about 2.0 nM, about 0.3 to about 1.5 nM, about 0.2 to about 1.0 nM or about 0.3 to about 1.0 nM.

In an embodiment of any preceding aspect of the invention, the variable domain neutralises rhesus IL-13 in a standard HEK STAT assay with an EC₅₀ of about 1 to about 20 nM, optionally about 1 to about 15 nM, about 2 to about 15 nM or about 2 to about 11.5 nM. In one aspect, the invention provides a single variable domain of the invention for neutralising rhesus IL-13 in a standard HEK STAT assay with an EC₅₀ of about 1 to about 20 nM, optionally about 1 to about 15 nM, about 2 to about 15 nM or about 2 to about 11.5 nM. In one aspect, the invention provides the use of a single variable domain of the invention in the manufacture of an IL-13 antagonist, wherein the variable domain or antagonist neutralises rhesus IL-13 in a standard HEK STAT assay with an EC₅₀ of about 1 to about 20 nM, optionally about 1 to about 15 nM, about 2 to about 15 nM or about 2 to about 11.5 nM.

In an embodiment of any preceding aspect of the invention, the variable domain neutralises cynomolgus monkey IL-13 in a standard HEK STAT assay with an ECₛₒ of about 1 to about 20 nM, optionally about 5 to 15 nM or about 5 to about 10 nM. In one aspect, the invention provides a single variable domain of the invention for neutralising cynomolgus monkey IL-13 in a standard HEK STAT assay with an EC₅₀ of about 1 to about 20 nM, optionally about 5 to 15 nM or about 5 to about 10 nM. In one aspect, the invention provides the use of a single variable domain of the invention in the manufacture of an IL-13 antagonist, wherein the variable domain or antagonist neutralises cynomolgus monkey IL-13 in a standard HEK STAT assay with an EC₅₀ of about 1 to about 20 nM, optionally about 5 to 15 nM or about 5 to about 10 nM.

An example of a standard HEK STAT assay is as follows: —
(i) anti-IL-13 dAb (or antagonist comprising an anti-IL-13 dAb) is incubated with 6 ng/ml IL-13 for one hour at 37° C., 5% CO₂;
(ii) The incubation mixture is added to 5×10⁴ HEKBlue™-STAT6 cells (HEK293 cells transfected with the STAT6 gene and the secreted embryonic alkaline phosphatase (SEAP) reporter gene) per well in DMEM (Dulbecco's modified Eagle medium) in a microtire plate;
(iii) The plate is incubated for 24 hours at 37 degrees centrigrade, 5% carbon dioxide;
(iv) The reporter gene product is detected and quantified.
In step (i), optionally the pre-incubation is performed using an equal volume of dAb and recombinant IL13. Optionally, dAb is titrated using ½ log dilutions from a top concentration of 400 nM (2× final concentration in the assay) to produce an 8-point dose-response curve, this is then incubated with the Il-13. In step (iv), optionally the culture supernatant is mixed with a QuantiBlue (Invivogen) and absorbance is read at 640 nm.
Anti-IL-13 dAb activity causes a decrease in STAT6 activation and a corresponding decrease in A₆₄₀ compared to IL-13 stimulation. EC₅₀ values can be calculated using techniques know to the skilled person.

“HEK 293 cells” refers to the human embryo kidney cell line designated 293 (ATCC Number CRL-1573) or its derivatives. For example, 293/SF cells (ATCC Number CRL-1573.1) are HEK 293 cells which have been adapted to grow in serum-free media. Also contemplated in this invention are HEK 293 cells adapted to grow in other culture conditions, or any kind of HEK 293 cells or derivatives. HEK-Blue™ STAT6 cells stably express the reporter gene secreted embryonic alkaline phosphatase (SEAP) under the control of the IFNβ minimal promoter fused to four STAT6 binding sites. See also Loignon et al, “Stable high volumetric production of glycosylated human recombinant IFNalpha2b in HEK293 cells”, BMC Biotechnology 2008, 8:65 for details of engineering of HEK293 cells and suitable constructs that can be adapted to engineer HEK293 cells transfected with the STAT6 gene and the secreted embryonic alkaline phosphatase (SEAP) report gene.

In an aspect, the invention provides an interleukin-13 (IL-13) antagonist comprising an anti-IL-13 immunoglobulin single variable domain according to the invention. Optionally, the antagonist does not consist of DOM10-53-616 (SEQ ID NO: 5). Optionally, the antagonist does not comprise DOM10-53-616 (SEQ ID NO: 5). Optionally, the antagonist does not comprise DOM10-53-616 (SEQ ID NO: 5) linked to a monoclonal antibody (mAb), optionally wherein the mAb is an anti-IL-4 mAb or an anti-IL-5 mAb.

In one embodiment, the antagonist competes with DOM10-53-546 (SEQ ID NO: 2); DOM10-53-567 (SEQ ID NO: 3); DOM10-53-568 (SEQ ID NO: 4); or DOM10-53-616 (SEQ ID NO: 5) for binding to IL-13. Optionally, the antagonist does not consist of DOM10-53-616 (SEQ ID NO: 5). Optionally, the antagonist does not comprise DOM10-53-616 (SEQ ID NO: 5). Optionally, the antagonist does not comprise DOM10-53-616 (SEQ ID NO: 5) linked to a monoclonal antibody (mAb), optionally wherein the mAb is an anti-IL-4 mAb or an anti-IL-5 mAb. In one embodiment, the IL-13 is human IL-13. In another embodiment, the IL-13 is Cynomolgus monkey IL-13. In another embodiment, the IL-13 is rhesus IL-13. In one embodiment, the antagonist competes with DOM10-53-546 (SEQ ID NO: 2); DOM10-53-567 (SEQ ID NO: 3); DOM10-53-568 (SEQ ID NO: 4); or DOM10-53-616 (SEQ ID NO: 5) for binding to human IL-13 and Cynomolgous monkey IL-13.

In another embodiment, the antagonist competes with DOM10-53-546 (SEQ ID NO: 2); DOM10-53-567 (SEQ ID NO: 3); DOM10-53-568 (SEQ ID NO: 4); or DOM10-53-616 (SEQ ID NO: 5) for binding to human IL-13, Cynomolgous monkey IL-13 and rhesus IL-13. Optionally, the antagonist does not consist of DOM10-53-616 (SEQ ID NO: 5). Optionally, the antagonist does not comprise DOM10-53-616 (SEQ ID NO: 5). Optionally, the antagonist does not comprise DOM10-53-616 (SEQ ID NO: 5) linked to a monoclonal antibody (mAb), optionally wherein the mAb is an anti-IL-4 mAb or an anti-IL-5 mAb.

In one aspect, the invention provides any anti-IL-13 single variable domain (eg, DOM10-53-616) or antagonist, composition or fusion protein according to the invention for pulmonary delivery. In one aspect, the invention provides the anti-IL-13 single variable domain or antagonist or fusion protein for delivery to the lung of a patient. In one aspect, the invention provides the use of any anti-IL-13 single variable domain (eg, DOM10-53-616) or antagonist, composition or fusion protein according to the invention in the manufacture of a medicament for pulmonary delivery. In one aspect, the invention provides the use of any anti-IL-13 single variable domain (eg, DOM10-53-616) or antagonist, composition or fusion protein according to the invention in the manufacture of a medicament for delivery to the lung of a patient. In one embodiment, the variable domain per se or when part of the antagonist or fusion protein is resistant to leucozyme and/or trypsin.

The invention provides a method for treating, suppressing or preventing other pulmonary diseases, for example chronic obstructive pulmonary disease (COPD) or pneumonia. Other pulmonary diseases that can be treated, suppressed or prevented in accordance with the invention include, for example, cystic fibrosis and asthma (e.g, steroid resistant asthma). Thus, in another aspect, the invention is a method for treating, suppressing or preventing a pulmonary disease (e.g, cystic fibrosis, asthma) comprising administering to a mammal in need thereof a therapeutically-effective dose or amount of a polypeptide, fusion protein, single variable domain (eg, DOM10-53-616), antagonist or composition according to the invention.

In particular embodiments, the polypeptide, fusion protein, single variable domain (eg, DOM10-53-616), antagonist or composition is administered via pulmonary delivery, such as by inhalation (e.g, intrabronchial, intranasal or oral inhalation, intranasal drops) or by systemic delivery (e.g, parenteral, intravenous, intramuscular, intraperitoneal, subcutaneous).

In a further aspect of the invention, there is provided a composition comprising any polypeptide, single variable domain(eg, DOM10-53-616), composition or antagonist according to the invention and a pharmaceutically acceptable carrier, diluent or excipient.

Moreover, the present invention provides a method for the treatment of disease using any polypeptide, single variable domain (eg, DOM10-53-616), composition or antagonist according to the present invention. In an embodiment the disease is cancer or an inflammatory disease, eg rheumatoid arthritis, asthma or Crohn's disease.

In an aspect of the invention, any polypeptide, single variable domain (eg, DOM10-53-616), composition or antagonist according to the invention is provided for therapy and/or prophylaxis of an IL-13-mediated condition in a human. In another aspect, there is provided the use of the polypeptide, single variable domain, composition or antagonist, in the manufacture of a medicament for therapy or prophylaxis of an IL-13-mediated condition in a human. In another aspect, there is provided a method of treating and/or preventing an IL-13-mediated condition in a human patient, the method comprising administering any polypeptide, single variable domain (eg, DOM10-53-616), composition or antagonist according to the invention to the patient. In one embodiment, the IL-13-mediated condition is a respiratory condition. In one embodiment, the IL-13-mediated condition is selected from lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

An aspect of the invention provides a pulmonary delivery device containing a polypeptide, single variable domain (eg, DOM10-53-616), composition or antagonist according to the invention. The device can be an inhaler or an intranasal administration device.

The ligand (e.g., polypeptide, single variable domain, composition or antagonist) of the invention can inhibit binding of IL-13 to IL-13Rα1 and/or IL-13Rα2, inhibit the activity of IL-13, and/or inhibit the activity of IL-13 without substantially inhibiting binding of IL-13 to IL-13Rα1 and/or IL-13Rα2. In one aspect, the invention provides a single variable domain according to the invention (eg, DOM10-53-616) for inhibiting binding of IL-13 to IL-13Rα1 and/or IL-13Rα2, inhibit the activity of IL-13, and/or inhibit the activity of IL-13 without substantially inhibiting binding of IL-13 to IL-13Rα1 and/or IL-13Rα2. In one aspect, the invention provides the use of a single variable domain according to the invention (eg, DOM10-53-616) in the manufacture of an IL-13 antagonist for inhibiting binding of IL-13 to IL-13Rα1 and/or IL-13Rα2, inhibit the activity of IL-13, and/or inhibit the activity of IL-13 without substantially inhibiting binding of IL-13 to IL-13Rα1 and/or IL-13Rα2.

In one embodiment, the ligand (e.g., immunoglobulin single variable domain) that binds IL-13 inhibits binding of IL-13 to an IL-13 receptor (e.g., IL-13Rα1, IL-13Rα2) with an inhibitory concentration 50 (IC₅₀) that is <about 10 μM, <about 1 μM, <about 100 nM, <about 10 nM, <about 1 nM, <about 500 pM, <about 300 pM, <about 100 pM, or <about 10 pM. The IC₅₀ is optionally determined using an in vitro receptor binding assay, such as the assay described herein.

It is also contemplated that the ligand (e.g., immunoglobulin single variable domain) optionally inhibit IL-13 induced functions in a suitable in vitro assay with a neutralizing dose 50 (ND50) that is <about 10 μM, <about 1 μM, <about 100 nM, <about 10 nM, <about 1 nM, <about 500 pM, <about 300 pM, <about 100 pM, <about 10 pM, <about 1 pM <about 500 fM, <about 300 fM, <about 100 fM, <about 10 fM. For example, the ligand can inhibit IL-13 induced proliferation of TF-1 cells (ATCC Accession No. CRL-2003) in an in vitro assay, such as the assay described herein wherein TF-1 cells were mixed with 5 ng/ml final concentration of IL-13.

It is contemplated that the ligand optionally inhibits IL-13 induced B cell proliferation by at least about 50%, at least about 60%, at least about 70%, at least about 80%, or at least about 90% in an in vitro assay, such as the assay described herein where 1×10⁵ B cells were incubated with 10 or 100 nM anti-IL-13 dAbs.

In an aspect of the invention, there is provided a dual-specific ligand comprising a single variable domain according to the invention. In more particular embodiments, the ligand has binding specificity for IL-4 and for IL-13 and comprises an immunoglobulin single variable domain with binding specificity for IL-4 which competes for binding to IL-4 with an anti-IL-4 domain antibody (dAb) selected from the group of anti-IL-4 dAbs disclosed in WO2007085815, the sequences of such dAbs being incorporated herein in their entirety for application to a dual-specific ligand according to the invention. Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

In all aspects of the invention, the or each immunoglobulin single variable domain is independently selected from antibody heavy chain and light chain single variable domains, eg V_{H}, V_{L} and V_{HH}.

In some embodiments, the dual-specific ligand can be an IgG-like format comprising two immunoglobulin single variable domains with binding specificity for IL-13 (e.g., two such domains that are identical to one another), and two immunoglobulin single variable domains with binding specificity for another target, eg IL-4. Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

In some embodiments, the dual-specific ligand can comprise an antibody Fc region. Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

In some embodiments, the dual-specific ligand can comprise an IgG constant region. Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

In other embodiments, any of the ligands described herein (eg., antagonist or single variable domain) further comprises a half-life extending moiety, such as a polyalkylene glycol moiety, serum albumin or a fragment thereof, transferrin receptor or a transferrin-binding portion thereof, or a moiety comprising a binding site for a polypeptide that enhance half-life in vivo. In some embodiments, the half-life extending moiety is a moiety comprising a binding site for a polypeptide that enhances half-life in vivo selected from the group consisting of an affibody, a SpA domain, an LDL receptor class A domain, an EGF domain, and an avimer.

In other embodiments, the half-life extending moiety is a polyethylene glycol moiety. In one embodiment, the antagonist comprises (optionally consists of) a single variable domain of the invention linked to a polyethylene glycol moiety (optionally, wherein said moiety has a size of about 20 to about 50 kDa, optionally about 40 kDa linear or branched PEG). Reference is made to WO04081026 for more detail on PEGylation of dAbs and binding moieties. In one embodiment, the antagonist consists of a dAb monomer linked to a PEG, wherein the dAb monomer is a single variable domain according to the invention, optionally DOM10-53-546 (SEQ ID NO: 2); DOM10-53-567 (SEQ ID NO: 3); DOM10-53-568 (SEQ ID NO: 4); or DOM10-53-616 (SEQ ID NO: 5). This antagonist can be provided for treatment of inflammatory disease, a lung condition (e.g., asthma, influenza or COPD) or cancer and optionally is for intravenous administration.

In other embodiments, the half-life extending moiety is an antibody or antibody fragment (e.g, an immunoglobulin single variable domain) comprising a binding site for serum albumin or neonatal Fc receptor.

The invention also relates to a ligand of the invention (eg., antagonist, or single variable domain) for use in therapy or diagnosis, and to the use of a ligand of the invention for the manufacture of a medicament for treatment, prevention or suppression of a disease described herein (e.g, allergic disease, Th2-mediated disease, asthma, cancer). Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

The invention also relates to a ligand of the invention (eg., antagonist, or single variable domain) for use in treating, suppressing or preventing a Th2-type immune response. Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

The invention also relates to therapeutic methods that comprise administering a therapeutically effective amount of a ligand of the invention (eg., antagonist, or single variable domain) to a subject in need thereof. In one embodiment, the invention relates to a method for inhibiting a Th2-type immune response comprising administering to a subject in need thereof a therapeutically effective amount of a ligand of the invention. Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

In other embodiments, the invention relates to a method for treating asthma comprising administering to a subject in need thereof a therapeutically effective amount of a ligand of the invention (eg., antagonist, or single variable domain). Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

In other embodiments, the invention relates to a method for treating cancer comprising administering to a subject in need thereof a therapeutically effective amount of a ligand of the invention (eg., antagonist, or single variable domain). Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

The invention also relates to a composition (e.g, pharmaceutical composition) comprising a ligand of the invention (eg., antagonist, or single variable domain) and a physiologically acceptable carrier. In some embodiments, the composition comprises a vehicle for intravenous, intramuscular, intraperitoneal, intraarterial, intrathecal, intraarticular, subcutaneous administration, pulmonary, intranasal, vaginal, or rectal administration.

The invention also relates to a drug delivery device comprising the composition (e.g, pharmaceutical composition) of the invention. In some embodiments, the drug delivery device comprises a plurality of therapeutically effective doses of ligand.

In other embodiments, the drug delivery device is selected from the group consisting of parenteral delivery device, intravenous delivery device, intramuscular delivery device, intraperitoneal delivery device, transdermal delivery device, pulmonary delivery device, intraarterial delivery device, intrathecal delivery device, intraarticular delivery device, subcutaneous delivery device, intranasal delivery device, vaginal delivery device, rectal delivery device, syringe, a transdermal delivery device, a capsule, a tablet, a nebulizer, an inhaler, an atomizer, an aerosolizer, a mister, a dry powder inhaler, a metered dose inhaler, a metered dose sprayer, a metered dose mister, a metered dose atomizer, and a catheter.

The ligands of the invention provide several advantages. For example, as described herein, the ligand can be tailored to have a desired in vivo serum half-life. Domain antibodies are much smaller than conventional antibodies, and can be administered to achieve better tissue penetration than conventional antibodies. Thus, dAbs and ligands that comprise a dAb provide advantages over conventional antibodies when administered to treat disease, such as Th2-mediated disease, asthma, allergic diseases, cancer (e.g, renal cell cancer). For example, asthma (e.g, allergic asthma) can be IgE-mediated or non-IgE-mediated, and ligands that have binding specificity for IL-4, IL-13 or IL-4 and IL-13 can be administered to treat both IgE-mediated and non-IgE-mediated asthma.

Similarly, due to the overlap and similarity in the biological activity of IL-4 and IL-13, therapy with ligands that have binding specificity for IL-4 and IL-13 can be administered to a patient (e.g, a patient with allergic disease (e.g, allergic asthma)) to provide superior therapy using a single therapeutic agent.

The ligand of the invention can be formatted as described herein. For example, the ligand of the invention can be formatted to tailor in vivo serum half-life. If desired, the ligand can further comprise a toxin or a toxin moiety as described herein. In some embodiments, the ligand comprises a surface active toxin, such as a free radical generator (e.g, selenium containing toxin) or a radionuclide. In other embodiments, the toxin or toxin moiety is a polypeptide domain (e.g, a dAb) having a binding site with binding specificity for an intracellular target. In particular embodiments, the ligand is an IgG-like format that has binding specificity for IL-13 (e.g, human IL-13).

The invention also relates to a method of inhibiting proliferation of peripheral blood mononuclear cells (PBMC) in an allergen-sensitized subject, comprising administering to a subject a pharmaceutical composition comprising any of the ligands of the invention (e.g., antagonist or single variable domain). In some embodiments, the allergen is selected from house dust mite, cat allergen, grass allergen, mold allergen, and pollen allergen.

The invention also relates to a method of inhibiting proliferation of B cells in a subject, comprising administering to the subject a pharmaceutical composition comprising a ligand of the invention. Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

The invention also relates to a pharmaceutical composition for treating preventing or suppressing a disease as described herein (e.g, Th2-mediated disease, allergic disease, asthma, cancer), comprising as an active ingredient a ligand as described herein. Optionally, the ligand does not consist of DOM10-53-616 (SEQ ID NO: 5). Optionally, the ligand does not comprise DOM10-53-616 (SEQ ID NO: 5). Optionally, the ligand does not comprise DOM10-53-616 (SEQ ID NO: 5) linked to a monoclonal antibody (mAb), optionally wherein the mAb is an anti-IL-4 mAb or an anti-IL-5 mAb.

In an aspect, the invention provides a fusion protein comprising the single variable domain of the invention. The variable domain can be fused, for example, to a peptide or polypeptide or protein. In one embodiment, the variable domain is fused to an antibody or antibody fragment, eg a monoclonal antibody. Generally, fusion can be achieved by expressing the fusion product from a single nucleic acid sequence or by expressing a polypeptide comprising the single variable domain and then assembling this polypeptide into a larger protein or antibody format using techniques that are conventional. Optionally, the fusion protein does not consist of DOM10-53-616 (SEQ ID NO: 5). Optionally, the fusion protein does not comprise DOM10-53-616 (SEQ ID NO: 5). Optionally, the fusion protein does not comprise DOM10-53-616 (SEQ ID NO: 5) linked to a monoclonal antibody (mAb), optionally wherein the mAb is an anti-IL-4 mAb or an anti-IL-5 mAb.

In one embodiment, the immunoglobulin single variable domain, antagonist or the fusion protein comprises an antibody constant domain. In one embodiment, the immunoglobulin single variable domain, antagonist or the fusion protein comprises an antibody Fc, optionally wherein the N-terminus of the Fc is linked (optionally directly linked) to the C-terminus of the variable domain. In one embodiment, the immunoglobulin single variable domain, antagonist or the fusion protein comprises comprises a half-life extending moiety. The half-life extending moiety can be a polyethylene glycol moiety, serum albumin or a fragment thereof, transferrin receptor or a transferrin-binding portion thereof, or an antibody or antibody fragment comprising a binding site for a polypeptide that enhances half-life in vivo. The half-life extending moiety can be an antibody or antibody fragment comprising a binding site for serum albumin or neonatal Fc receptor. The half-life extending moiety can be a dAb, antibody or antibody fragment. In one embodiment, the immunoglobulin single variable domain or the antagonist or the fusion protein is provided such that the variable domain (or the variable domain comprised by the antagonist or fusion protein) further comprises a polyalkylene glycol moiety. The polyalkylene glycol moiety can be a polyethylene glycol moiety. Further discussion is provided below.

In one embodiment, the single variable domain of the invention binds human IL-13 with a dissociation constant (Kd) of about 10 to about 150 pM, optionally about 50 to about 150 pM, optionally about 70 to about 150 pM, as determined by surface plasmon resonance. In one aspect, the invention provides a single variable domain of the invention for binding human IL-13 with a dissociation constant (Kd) of about 10 to about 150 pM, optionally about 50 to about 150 pM, optionally about 70 to about 150 pM, as determined by surface plasmon resonance. In one aspect, the invention provides the use of a single variable domain of the invention in the manufacture of an IL-13 antagonist, wherein the variable domain or antagonist binds human IL-13 with a dissociation constant (Kd) of about 10 to about 150 pM, optionally about 50 to about 150 pM, optionally about 70 to about 150 pM, as determined by surface plasmon resonance.

In one embodiment, the single variable domain of the invention binds Cynomolgus monkey IL-13 with a dissociation constant (Kd) of about 1 to about 5 nM, as determined by surface plasmon resonance. In one aspect, the invention provides a single variable domain of the invention for binding Cynomolgus monkey IL-13 with a dissociation constant (Kd) of about 1 to about 5 nM, as determined by surface plasmon resonance. In one aspect, the invention provides the use of a single variable domain of the invention in the manufacture of an IL-13 antagonist, wherein the variable domain or antagonist binds Cynomolgus monkey IL-13 with a dissociation constant (Kd) of about 1 to about 5 nM, as determined by surface plasmon resonance.

In one embodiment, the single variable domain of the invention (eg, DOM10-53-616) is provided as a dAb monomer, optionally unformatted (e.g., not PEGylated or half-life extended) or linked to a PE.G., optionally as a dry powder formulation, optionally for delivery to a patient by inhalation (e.g., pulmonary delivery), optionally for treating and/or preventing a lung condition (e.g., asthma, COPD or influenza). In one embodiment, the single variable domain of the invention is provided as dAb monomer (not PEGylated or half-life extended) for delivery to a patient by inhalation (e.g., pulmonary delivery), optionally for treating and/or preventing a lung condition (e.g., asthma, COPD or influenza).

In one aspect, the present invention provides the single variable domain (eg, DOM10-53-616), protein, polypeptide, antagonist, composition or device of any aspect or embodiment of the invention for providing one or more of the following (an explicit combination of two or more of the following purposes is hereby disclosed and can be the subject of a claim):—
(i) Potent binding of human IL-13 (e.g., with a dissociation constant (Kd) of about 1 nM or less, optionally about 500 pM or less, optionally about 250 pM or less, optionally about 150 pM or less, optionally about 100 pM or less, optionally about 1 nM to about 10, about 50 or about 70 pM, optionally about 500 to about 10, about 50 or about 70 pM, optionally about 250 to about 10, about 50 or about 70 pM, optionally about 150 to about 10, about 50 or about 70 pM, or optionally about 100 to about 10, about 50 or about 70 pM);
(ii) Potent binding of a non-human primate IL-13 (e.g., Cynomolgus monkey, rhesus or baboon IL-13) (e.g., with a dissociation constant (Kd) of about 5 nM or less, optionally about 4, about 3, about 2 or about 1 nM or less, optionally about 1 to about 5 nM);
(iii) Potent binding of human IL-13 (e.g., with a dissociation constant (Kd) of about 1 nM or less, optionally about 500 pM or less, optionally about 250 pM or less, optionally about 150 pM or less, optionally about 100 pM or less, optionally about 1 nM to about 10, about 50 or about 70 pM, optionally about 500 to about 10, about 50 or about 70 pM, optionally about 250 to about 10, about 50 or about 70 pM, optionally about 150 to about 10, about 50 or about 70 pM, or optionally about 100 to about 10, about 50 or about 70 pM) and potent binding of a non-human primate IL-13 (e.g., Cynomolgus monkey, rhesus or baboon IL-13) (e.g., with a dissociation constant (Kd) of about 5 nM or less, optionally about 4, about 3, about 2 or about 1 nM or less, optionally about 1 to about 5 nM);
(iv) Potent binding of human, Cynomolgus monkey and rhesus IL-13 (e.g., binding human IL-13 with a dissociation constant (Kd) of about 1 nM or less, optionally about 500 pM or less, optionally about 250 pM or less, optionally about 150 pM or less, optionally about 100 pM or less, optionally about 1 nM to about 10, about 50 or about 70 pM, optionally about 500 to about 10, about 50 or about 70 pM, optionally about 250 to about 10, about 50 or about 70 pM, optionally about 150 to about 10, about 50 or about 70 pM, or optionally about 100 to about 10, about 50 or about 70 pM; and binding each of Cynomolgus monkey and rhesus IL-13 with a dissociation constant (Kd) of about 5 nM or less, optionally about 4, about 3, about 2 or about 1 nM or less, optionally about 1 to about 5 nM);
(v) Good expression in prokaryotic cells (e.g. Expression in prokaryotic cells (e.g., E. coli) of at least about 3 mg/litre);
(vi) Potent neutralization of human IL-13 in a patient, e.g., neutralization using a single variable domain, protein, polypeptide, antagonist or composition that neutralises human IL-13 in a standard HEK STAT assay with an EC₅₀ of about 0.1 to about 2.0 nM, optionally about 0.2 to about 2.0 nM, about 0.3 to about 1.5 nM, about 0.2 to about 1.0 nM or about 0.3 to about 1.0 nM;
(vii) Potent neutralization of human IL-13 in a patient, e.g., neutralization using a single variable domain, protein, polypeptide, antagonist or composition that neutralises Cynomolgus monkey IL-13 in a standard HEK STAT assay with an EC₅₀ of about 1 to about 20 nM, optionally about 5 to about 15 nM or about 5 to about 10 nM;
(viii) Potent neutralization of human IL-13 in a patient, e.g., neutralization using a single variable domain, protein, polypeptide, antagonist or composition that neutralises rhesus IL-13 in a standard HEK STAT assay with an EC₅₀ of about 1 to about 15 nM, about 2 to about 15 nM or about 2 to about 11.5 nM;
(ix) Potent neutralization of human IL-13 and non-human primate, e.g., neutralization using a single variable domain, protein, polypeptide, antagonist or composition that neutralises human IL-13 in a standard HEK STAT assay with an EC₅₀ of about 0.1 to about 2.0 nM, optionally about 0.2 to about 2.0 nM, about 0.3 to about 1.5 nM, about 0.2 to about 1.0 nM or about 0.3 to about 1.0 nM; and neutralises non-human primate IL-13 in a standard HEK STAT assay with an EC₅₀ of about 1 to about 20 nM, optionally about 5 to about 15 nM or about 5 to about 10 nM;
(x) Providing cross-reactivity between more than one species of primate IL-13 (optionally, human and Cynomolgus monkey and/or rhesus IL-13, e.g., human and Cynomolgus monkey IL-13 or human and rhesus IL-13 or human and baboon IL-13); and
(xi) Providing protease stability (optionally, trypsin stability).

In one aspect, the present invention provides the use of the single variable domain (eg, DOM10-53-616), protein, polypeptide, antagonist, composition or device of any aspect or embodiment of the invention for providing one or more of (i) to (xi) in the immediately preceding paragraph. The invention also provides corresponding methods.

Reference is made to WO2007085815, which discloses anti-IL-13 immunoglobulin single variable domains. The disclosure of this document is incorporated herein in its entirety, in particular to provide for uses, formats, methods of selection, methods of production, methods of formulation and assays for anti-IL-13 single variable domains, ligands, antagonists and the like, so that these disclosures can be applied specifically and explicitly in the context of the present invention, including to provide explicit description for importation into claims of the present disclosure.

The anti-IL-13 is an immunoglobulin single variable domain can be any suitable immunoglobulin variable domain, and optionally is a human variable domain or a variable domain that comprises or are derived from human framework regions (e.g., DP47 or DPK9 framework regions). In certain embodiments, the variable domain is based on a universal framework, as described herein.

In certain embodiments, a polypeptide domain (e.g., immunoglobulin single variable domain) that has a binding site with binding specificity for IL-13 resists aggregation, unfolds reversibly (see WO04101790, the teachings of which are incorporated herein by reference).

### Nucleic Acid Molecules, Vectors and Host Cells

The invention also provides isolated and/or recombinant nucleic acid molecules encoding ligands (single variable domains, fusion proteins, polypeptides, dual-specific ligands and multispecific ligands) as described herein.

In one aspect, the invention provides an isolated or recombinant nucleic acid encoding a polypeptide comprising an immunoglobulin single variable domain according to the invention. In one embodiment, the nucleic acid comprises the nucleotide sequence of DOM10-53-546 (SEQ ID NO: 6); DOM10-53-567 (SEQ ID NO: 7); DOM10-53-568 (SEQ ID NO: 8); or DOM10-53-616 (SEQ ID NO: 9). In one embodiment, the nucleic acid comprises the nucleotide sequence of DOM10-53-546 (SEQ ID NO: 6); DOM10-53-567 (SEQ ID NO: 7) or DOM10-53-568 (SEQ ID NO: 8).

In one aspect, the invention provides an isolated or recombinant nucleic acid, wherein the nucleic acid comprises a nucleotide sequence that is at least 99% identical to the nucleotide sequence of DOM10-53-546 (SEQ ID NO: 6); DOM10-53-567 (SEQ ID NO: 7); DOM10-53-568 (SEQ ID NO: 8); or DOM10-53-616 (SEQ ID NO: 9), and wherein the nucleic acid encodes a polypeptide comprising an immunoglobulin single variable domain that specifically binds to IL-13. Optionally, the nucleic acid does not consist of or comprise the nucleotide sequence DOM10-53-616 (SEQ ID NO: 9).

In one aspect, the invention provides a vector comprising a nucleic acid of the invention. In one aspect, the invention provides a host cell comprising a nucleic acid of the invention or the vector. There is provided a method of producing polypeptide comprising an immunoglobulin single variable domain, the method comprising maintaining the host cell under conditions suitable for expression of said nucleic acid or vector, whereby a polypeptide comprising an immunoglobulin single variable domain is produced. Optionally, the method further comprises the step of isolating the polypeptide and optionally producing a variant, eg a mutated variant, having an improved affinity (Kd); EC₅₀ for IL-13 neutralization in a standard HEK STAT assay than the isolated polypeptide.

Nucleic acids referred to herein as “isolated” are nucleic acids which have been separated away from the nucleic acids of the genomic DNA or cellular RNA of their source of origin (e.g., as it exists in cells or in a mixture of nucleic acids such as a library), and include nucleic acids obtained by methods described herein or other suitable methods, including essentially pure nucleic acids, nucleic acids produced by chemical synthesis, by combinations of biological and chemical methods, and recombinant nucleic acids which are isolated (see e.g., Daugherty, B. L. et al., Nucleic Acids Res., 19(9): 2471-2476 (1991); Lewis, A. P. and J. S. Crowe, Gene, 101: 297-302 (1991)).

Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes.

In certain embodiments, the isolated and/or recombinant nucleic acid comprises a nucleotide sequence encoding a ligand, as described herein, wherein said ligand comprises an amino acid sequence that has at least about 80%, at least about 85%, at least about 90%, at least about 91%, at least about 92%, at least about 93%, at least about 94%, at least about 95%, at least about 96%, at least about 97%, at least about 98%, or at least about 99% amino acid sequence identity with the amino acid sequence of a dAb that binds IL-13 disclosed herein, eg DOM10-53-546 (SEQ ID NO: 2); DOM10-53-567 (SEQ ID NO: 3); DOM10-53-568 (SEQ ID NO: 4); or DOM10-53-616 (SEQ ID NO: 5). Nucleotide sequence identity can be determined over the whole length of the nucleotide sequence that encodes the selected anti-IL-13 dAb.

The invention also provides a vector comprising a recombinant nucleic acid molecule of the invention. In certain embodiments, the vector is an expression vector comprising one or more expression control elements or sequences that are operably linked to the recombinant nucleic acid of the invention The invention also provides a recombinant host cell comprising a recombinant nucleic acid molecule or vector of the invention. Suitable vectors (e.g, plasmids, phagmids), expression control elements, host cells and methods for producing recombinant host cells of the invention are well-known in the art, and examples are further described herein.

Suitable expression vectors can contain a number of components, for example, an origin of replication, a selectable marker gene, one or more expression control elements, such as a transcription control element (e.g, promoter, enhancer, terminator) and/or one or more translation signals, a signal sequence or leader sequence, and the like. Expression control elements and a signal sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for prokaryotic (e.g, lac, tac, T3, T7 promoters for E. coli) and eukaryotic (e.g, Simian Virus 40 early or late promoter, Rous sarcoma virus long terminal repeat promoter, cytomegalovirus promoter, adenovirus late promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in prokaryotic (e.g, lactamase gene (ampicillin resistance), Tet gene for tetracycline resistance) and eukaryotic cells (e.g, neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g, LEU2, URA3, HIS3) are often used as selectable markers in yeast. Use of viral (e.g, baculovirus) or phage vectors, and vectors which are capable of integrating into the genome of the host cell, such as retroviral vectors, are also contemplated. Suitable expression vectors for expression in mammalian cells and prokaryotic cells (E. coli), insect cells (Drosophila Schnieder S2 cells, Sf9) and yeast (P. methanolica, P. pastoris, S. cerevisiae) are well-known in the art.

Suitable host cells can be prokaryotic, including bacterial cells such as E. coli, B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acac. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NS0 cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In certain embodiments, the host cell is a non-human host cell.

The invention also provides a method for producing a ligand (e.g, dual-specific ligand, multispecific ligand) of the invention, comprising maintaining a recombinant host cell comprising a recombinant nucleic acid of the invention under conditions suitable for expression of the recombinant nucleic acid, whereby the recombinant nucleic acid is expressed and a ligand is produced. In some embodiments, the method further comprises isolating the ligand.

Reference is made to WO200708515, page 161, line 24 to page 189, line 10 for details of disclosure that is applicable to embodiments of the present invention. This disclosure is hereby incorporated herein by reference as though it appears explicitly in the text of the present disclosure and relates to the embodiments of the present invention, and to provide explicit support for disclosure to incorporated into claims below. This includes disclosure presented in WO200708515, page 161, line 24 to page 189, line 10 providing details of the “Preparation of Immunoglobulin Based Ligands”, “Library vector systems”, “Library Construction”, “Combining Single Variable Domains”, “Characterisation of Ligands”, “Structure of Ligands”, “Skeletons”, “Protein Scaffolds”, “Scaffolds for Use in Constructing Ligands”, “Diversification of the Canonical Sequence” and “Therapeutic and diagnostic compositions and uses”, as well as definitions of “operably linked”, “naive”, “prevention”, “suppression”, “treatment”, “allergic disease”, “Th2-mediated disease”, “therapeutically-effective dose” and “effective”.

### Formats

Increased half-life is useful in in vivo applications of immunoglobulins, especially antibodies and most especially antibody fragments of small size. Such fragments (Fvs, disulphide bonded Fvs, Fabs, scFvs, dAbs) suffer from rapid clearance from the body; thus, whilst they are able to reach most parts of the body rapidly, and are quick to produce and easier to handle, their in vivo applications have been limited by their only brief persistence in vivo. One embodiment of the invention solves this problem by providing increased half-life of the ligands in vivo and consequently longer persistence times in the body of the functional activity of the ligand.

Methods for pharmacokinetic analysis and determination of ligand half-life will be familiar to those skilled in the art. Details may be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2^{nd} Rev. ex edition (1982), which describes pharmacokinetic parameters such as t alpha and t beta half lives and area under the curve (AUC).

Half lives (t½ alpha and t½ beta) and AUC can be determined from a curve of serum concentration of ligand against time. The WinNonlin analysis package (available from Pharsight Corp., Mountain View, Calif. 94040, USA) can be used, for example, to model the curve. In a first phase (the alpha phase) the ligand is undergoing mainly distribution in the patient, with some elimination. A second phase (beta phase) is the terminal phase when the ligand has been distributed and the serum concentration is decreasing as the ligand is cleared from the patient. The t alpha half life is the half life of the first phase and the t beta half life is the half life of the second phase. Thus, in one embodiment, the present invention provides a ligand or a composition comprising a ligand according to the invention having a tα half-life in the range of 15 minutes or more. In one embodiment, the lower end of the range is 30 minutes, 45 minutes, 1 hour, 2 hours, 3 hours, 4 hours, 5 hours, 6 hours, 7 hours, 10 hours, 11 hours or 12 hours. In addition, or alternatively, a ligand or composition according to the invention will have a tα half life in the range of up to and including 12 hours. In one embodiment, the upper end of the range is 11, 10, 9, 8, 7, 6 or 5 hours. An example of a suitable range is 1 to 6 hours, 2 to 5 hours or 3 to 4 hours.

In one embodiment, the present invention provides a ligand (polypeptide, dAb or antagonist) or a composition comprising a ligand according to the invention having a tβ half-life in the range of about 2.5 hours or more. In one embodiment, the lower end of the range is about 3 hours, about 4 hours, about 5 hours, about 6 hours, about 7 hours, about 10 hours, about 11 hours, or about 12 hours. In addition, or alternatively, a ligand or composition according to the invention has a tβ half-life in the range of up to and including 21 days. In one embodiment, the upper end of the range is about 12 hours, about 24 hours, about 2 days, about 3 days, about 5 days, about 10 days, about 15 days or about 20 days. In one embodiment a ligand or composition according to the invention will have a tβ half life in the range about 12 to about 60 hours. In a further embodiment, it will be in the range about 12 to about 48 hours. In a further embodiment still, it will be in the range about 12 to about 26 hours.

In addition, or alternatively to the above criteria, the present invention provides a ligand or a composition comprising a ligand according to the invention having an AUC value (area under the curve) in the range of about 1 mg·min/ml or more. In one embodiment, the lower end of the range is about 5, about 10, about 15, about 20, about 30, about 100, about 200 or about 300 mg·min/ml. In addition, or alternatively, a ligand or composition according to the invention has an AUC in the range of up to about 600 mg·min/ml. In one embodiment, the upper end of the range is about 500, about 400, about 300, about 200, about 150, about 100, about 75 or about 50 mg·min/ml. In one embodiment a ligand according to the invention will have a AUC in the range selected from the group consisting of the following: about 15 to about 150 mg·min/ml, about 15 to about 100 mg·min/ml, about 15 to about 75 mg·min/ml, and about 15 to about 50 mg·min/ml.

Polypeptides and dAbs of the invention and antagonists comprising these can be formatted to have a larger hydrodynamic size, for example, by attachment of a PEG group, serum albumin, transferrin, transferrin receptor or at least the transferrin-binding portion thereof, an antibody Fc region, or by conjugation to an antibody domain. For example, polypeptides dAbs and antagonists formatted as a larger antigen-binding fragment of an antibody or as an antibody (e.g, formatted as a Fab, Fab′, F(ab)₂, F(ab′)₂, IgG, scFv).

Hydrodynamic size of the ligands (e.g, dAb monomers and multimers) of the invention may be determined using methods which are well known in the art. For example, gel filtration chromatography may be used to determine the hydrodynamic size of a ligand. Suitable gel filtration matrices for determining the hydrodynamic sizes of ligands, such as cross-linked agarose matrices, are well known and readily available.

The size of a ligand format (e.g, the size of a PEG moiety attached to a dAb monomer), can be varied depending on the desired application. For example, where ligand is intended to leave the circulation and enter into peripheral tissues, it is desirable to keep the hydrodynamic size of the ligand low to facilitate extravazation from the blood stream. Alternatively, where it is desired to have the ligand remain in the systemic circulation for a longer period of time the size of the ligand can be increased, for example by formatting as an Ig like protein.

Half-Life Extension by Targeting an Antigen or Epitope that Increases Half-Live In Vivo

The hydrodynamic size of a ligand and its serum half-life can also be increased by conjugating or associating an IL-13 binding polypeptide, dAb or antagonist of the invention to a binding domain (e.g, antibody or antibody fragment) that binds an antigen or epitope that increases half-live in vivo, as described herein. For example, the IL-13 binding agent (e.g, polypeptide) can be conjugated or linked to an anti-serum albumin or anti-neonatal Fc receptor antibody or antibody fragment, eg an anti-SA or anti-neonatal Fc receptor dAb, Fab, Fab′ or scFv, or to an anti-SA affibody or anti-neonatal Fc receptor Affibody or an anti-SA avimer, or an anti-SA binding domain which comprises a scaffold selected from, but not limited to, the group consisting of CTLA-4, lipocallin, SpA, an affibody, an avimer, GroEl and fibronectin (see WO2008096158 for disclosure of these binding domains, which domains and their sequences are incorporated herein by reference and form part of the disclosure of the present text). Conjugating refers to a composition comprising polypeptide, dAb or antagonist of the invention that is bonded (covalently or noncovalently) to a binding domain that binds serum albumin.

Suitable polypeptides that enhance serum half-life in vivo include, for example, transferrin receptor specific ligand-neuropharmaceutical agent fusion proteins (see U.S. Pat. No. 5,977,307, the teachings of which are incorporated herein by reference), brain capillary endothelial cell receptor, transferrin, transferrin receptor (e.g, soluble transferrin receptor), insulin, insulin-like growth factor 1 (IGF 1) receptor, insulin-like growth factor 2 (IGF 2) receptor, insulin receptor, blood coagulation factor X, α1-antitrypsin and HNF 1α. Suitable polypeptides that enhance serum half-life also include alpha-1 glycoprotein (orosomucoid; AAG), alpha-1 antichymotrypsin (ACT), alpha-1 microglobulin (protein HC; AIM), antithrombin III (AT III), apolipoprotein A-1 (Apo A-1), apolipoprotein B (Apo B), ceruloplasmin (Cp), complement component C3 (C3), complement component C4 (C4), C1 esterase inhibitor (C1 INH), C-reactive protein (CRP), ferritin (FER), hemopexin (HPX), lipoprotein(a) (Lp(a)), mannose-binding protein (MBP), myoglobin (Myo), prealbumin (transthyretin; PAL), retinol-binding protein (RBP), and rheumatoid factor (RF).

Suitable proteins from the extracellular matrix include, for example, collagens, laminins, integrins and fibronectin. Collagens are the major proteins of the extracellular matrix. About 15 types of collagen molecules are currently known, found in different parts of the body, e.g, type I collagen (accounting for 90% of body collagen) found in bone, skin, tendon, ligaments, cornea, internal organs or type II collagen found in cartilage, vertebral disc, notochord, and vitreous humor of the eye.

Suitable proteins from the blood include, for example, plasma proteins (e.g, fibrin, α-2 macroglobulin, serum albumin, fibrinogen (e.g, fibrinogen A, fibrinogen B), serum amyloid protein A, haptoglobin, profilin, ubiquitin, uteroglobulin and β-2-microglobulin), enzymes and enzyme inhibitors (e.g, plasminogen, lysozyme, cystatin C, alpha-1-antitrypsin and pancreatic trypsin inhibitor), proteins of the immune system, such as immunoglobulin proteins (e.g, IgA, IgD, IgE, IgG, IgM, immunoglobulin light chains (kappa/lambda)), transport proteins (e.g, retinol binding protein, α-1 microglobulin), defensins (e.g, beta-defensin 1, neutrophil defensin 1, neutrophil defensin 2 and neutrophil defensin 3) and the like.

Suitable proteins found at the blood brain barrier or in neural tissue include, for example, melanocortin receptor, myelin, ascorbate transporter and the like.

Suitable polypeptides that enhance serum half-life in vivo also include proteins localized to the kidney (e.g, polycystin, type IV collagen, organic anion transporter K1, Heymann's antigen), proteins localized to the liver (e.g, alcohol dehydrogenase, G250), proteins localized to the lung (e.g, secretory component, which binds IgA), proteins localized to the heart (e.g, HSP 27, which is associated with dilated cardiomyopathy), proteins localized to the skin (e.g, keratin), bone specific proteins such as morphogenic proteins (BMPs), which are a subset of the transforming growth factor β superfamily of proteins that demonstrate osteogenic activity (e.g, BMP-2, BMP-4, BMP-5, BMP-6, BMP-7, BMP-8), tumor specific proteins (e.g, trophoblast antigen, herceptin receptor, oestrogen receptor, cathepsins (e.g, cathepsin B, which can be found in liver and spleen)).

Suitable disease-specific proteins include, for example, antigens expressed only on activated T-cells, including LAG-3 (lymphocyte activation gene), osteoprotegerin ligand (OPGL; see Nature 402, 304-309 (1999)), OX40 (a member of the TNF receptor family, expressed on activated T cells and specifically up-regulated in human T cell leukemia virus type-I (HTLV-I)-producing cells; see Immunol. 165 (1):263-70 (2000)). Suitable disease-specific proteins also include, for example, metalloproteases (associated with arthritis/cancers) including CG6512 Drosophila, human paraplegin, human FtsH, human AFG3L2, murine ftsH; and angiogenic growth factors, including acidic fibroblast growth factor (FGF-1), basic fibroblast growth factor (FGF-2), vascular endothelial growth factor/vascular permeability factor (VEGF/VPF), transforming growth factor-α (TGF α), tumor necrosis factor-alpha (TNF-α), angiogenin, interleukin-3 (IL-3), interleukin-8 (IL-8), platelet-derived endothelial growth factor (PD-ECGF), placental growth factor (P1GF), midkine platelet-derived growth factor-BB (PDGF), and fractalkine.

Suitable polypeptides that enhance serum half-life in vivo also include stress proteins such as heat shock proteins (HSPs). HSPs are normally found intracellularly. When they are found extracellularly, it is an indicator that a cell has died and spilled out its contents. This unprogrammed cell death (necrosis) occurs when as a result of trauma, disease or injury, extracellular HSPs trigger a response from the immune system. Binding to extracellular HSP can result in localizing the compositions of the invention to a disease site.

Suitable proteins involved in Fc transport include, for example, Brambell receptor (also known as FcRB). This Fc receptor has two functions, both of which are potentially useful for delivery. The functions are (1) transport of IgG from mother to child across the placenta (2) protection of IgG from degradation thereby prolonging its serum half-life. It is thought that the receptor recycles IgG from endosomes. (See, Holliger et al, Nat Biotechnol 15(7):632-6 (1997).)

dAbs that Bind Serum Albumin

The invention in one embodiment provides a polypeptide or antagonist (e.g., dual specific ligand comprising an anti-IL-13 dAb (a first dAb)) that binds to IL-13 and a second dAb that binds serum albumin (SA), the second dAb binding SA with a Kd as determined by surface plasmon resonance of about 1 nM to about 1, about 2, about 3, about 4, about 5, about 10, about 20, about 30, about 40, about 50, about 60, about 70, about 100, about 200, about 300, about 400 or about 500 μM (i.e., ×10⁻⁹ to 5×10⁻⁴M), or about 100 nM to about 10 μM, or about 1 to about 5 μM or about 3 to about 70 nM or about 10 nM to about 1, about 2, about 3, about 4 or about 5 μM. For example about 30 to about 70 nM as determined by surface plasmon resonance. In one embodiment, the first dAb (or a dAb monomer) binds SA (e.g., USA) with a Kd as determined by surface plasmon resonance of approximately about 1, about 50, about 70, about 100, about 150, about 200, about 300 nM or about 1, about 2 or about 3 μM. In one embodiment, for a dual specific ligand comprising a first anti-SA dAb and a second dAb to IL-13, the affinity (e.g., Kd and/or K_{off} as measured by surface plasmon resonance, e.g., using BiaCore) of the second dAb for its target is from about 1 to about 100000 times (e.g., about 100 to about 100000, or about 1000 to about 100000, or about 10000 to about 100000 times) the affinity of the first dAb for SA. In one embodiment, the serum albumin is human serum albumin (HSA). For example, the first dAb binds SA with an affinity of approximately about 10 μM, while the second dAb binds its target with an affinity of about 100 pM. In one embodiment, the serum albumin is human serum albumin (HSA). In one embodiment, the first dAb binds SA (e.g., HSA) with a Kd of approximately about 50, for example about 70, about 100, about 150 or about 200 nM. Details of dual specific ligands are found in WO03002609, WO04003019, WO2008096158 and WO04058821.

The ligands of the invention can in one embodiment comprise a dAb that binds serum albumin (SA) with a Kd as determined by surface plasmon resonance of about 1 nM to about 1, about 2, about 3, about 4, about 5, about 10, about 20, about 30, about 40, about 50, about 60, about 70, about 100, about 200, about 300, about 400 or about 500 μM (i.e., x about 10⁻⁹ to about 5×10⁻⁴M), or about 100 nM to about 10 μM, or about 1 to about 5 μM or about 3 to about 70 nM or about 10 nM to about 1, about 2, about 3, about 4 or about 5 μM. For example about 30 to about 70 nM as determined by surface plasmon resonance. In one embodiment, the first dAb (or a dAb monomer) binds SA (e.g., HSA) with a Kd as determined by surface plasmon resonance of approximately about 1, about 50, about 70, about 100, about 150, about 200, about 300 nM or about 1, about 2 or about 3 μM. In one embodiment, the first and second dAbs are linked by a linker, for example a linker of from 1 to 4 amino acids or from 1 to 3 amino acids, or greater than 3 amino acids or greater than 4, 5, 6, 7, 8, 9, 10, 15 or 20 amino acids. In one embodiment, a longer linker (greater than 3 amino acids) is used to enhance potency (Kd of one or both dAbs in the antagonist).

In particular embodiments of the ligands and antagonists, the dAb binds human serum albumin and competes for binding to albumin with a dAb selected from the group consisting of
MSA-16, MSA-26 (See WO04003019 for disclosure of these sequences, which sequences and their nucleic acid counterpart are incorporated herein by reference and form part of the disclosure of the present text),
DOM7m-16(SEQ ID NO: 473), DOM7m-12(SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1(SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), DOM7r-33 (SEQ ID NO: 517) (See WO2007080392 for disclosure of these sequences, which sequences and their nucleic acid counterpart are incorporated herein by reference and form part of the disclosure of the present text; the SEQ ID No's in this paragraph are those that appear in WO2007080392),
dAb8 (dAb 10), dAb 10, dAb36, dAb7r20 (DOM7r20), dAb7r21 (DOM7r21), dAb7r22 (DOM7r22), dAb7r23 (DOM7r23), dAb7r24 (DOM7r24), dAb7r25 (DOM7r25), dAb7r26 (DOM7r26), dAb7r27 (DOM7r27), dAb7r28 (DOM7r28), dAb7r29 (DOM7r29), dAb7r29 (DOM7r29), dAb7r31 (DOM7r31), dAb7r32 (DOM7r32), dAb7r33 (DOM7r33), dAb7r33 (DOM7r33), dAb7h22 (DOM7h22), dAb7h23 (DOM7h23), dAb7h24 (DOM7h24), dAb7h25 (DOM7h25), dAb7h26 (DOM7h26), dAb7h27 (DOM7h27), dAb7h30 (DOM7h30), dAb7h31 (DOM7h31), dAb2 (dAbs 4, 7, 41), dAb4, dAb7, dAb 11, dAb12 (dAb7 m12), dAb13 (dAb 15), dAb15, dAb16 (dAb21, dAb7m16), dAb17, dAb18, dAb19, dAb21, dAb22, dAb23, dAb24, dAb25 (dAb26, dAb7m26), dAb27, dAb30 (dAb35), dAb31, dAb33, dAb34, dAb35, dAb38 (dAb54), dAb41, dAb46 (dAbs 47, 52 and 56), dAb47, dAb52, dAb53, dAb54, dAb55, dAb56, dAb7m12, dAb7m16, dAb7m26, dAb7r1 (DOM7r1), dAb7r3 (DOM7r3), dAb7r4 (DOM7r4), dAb7r5 (DOM7r5), dAb7r7 (DOM7r7), dAb7r8 (DOM7r8), dAb7r13 (DOM7r13), dAb7r14 (DOM7r14), dAb7r15 (DOM7r15), dAb7r16 (DOM7r16), dAb7r17 (DOM7r17), dAb7r18 (DOM7r18), dAb7r19 (DOM7r19), dAb7h1 (DOM7h1), dAb7h2 (DOM7h2), dAb7h6 (DOM7h6), dAb7h7 (DOM7h7), dAb7h8 (DOM7h8), dAb7h9 (DOM7h9), dAb7h10 (DOM7h10), dAb7h11 (DOM7h11), dAb7h12 (DOM7h12), dAb7h13 (DOM7h13), dAb7h14 (DOM7h14), dAb7p1 (DOM7p1), and dAb7p2 (DOM7p2) (see WO2008096158 for disclosure of these sequences, which sequences and their nucleic acid counterpart are incorporated herein by reference and form part of the disclosure of the present text). Alternative names are shown in brackets after the dAb, e.g, dAb8 has an alternative name which is dAb10 i.e. dAb8 (dAb10).

In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of
MSA-16, MSA-26,
DOM7m-16(SEQ ID NO: 473), DOM7m-12(SEQ ID NO: 474), DOM7m-26 (SEQ ID NO: 475), DOM7r-1 (SEQ ID NO: 476), DOM7r-3 (SEQ ID NO: 477), DOM7r-4 (SEQ ID NO: 478), DOM7r-5 (SEQ ID NO: 479), DOM7r-7 (SEQ ID NO: 480), DOM7r-8 (SEQ ID NO: 481), DOM7h-2 (SEQ ID NO: 482), DOM7h-3 (SEQ ID NO: 483), DOM7h-4 (SEQ ID NO: 484), DOM7h-6 (SEQ ID NO: 485), DOM7h-1(SEQ ID NO: 486), DOM7h-7 (SEQ ID NO: 487), DOM7h-22 (SEQ ID NO: 489), DOM7h-23 (SEQ ID NO: 490), DOM7h-24 (SEQ ID NO: 491), DOM7h-25 (SEQ ID NO: 492), DOM7h-26 (SEQ ID NO: 493), DOM7h-21 (SEQ ID NO: 494), DOM7h-27 (SEQ ID NO: 495), DOM7h-8 (SEQ ID NO: 496), DOM7r-13 (SEQ ID NO: 497), DOM7r-14 (SEQ ID NO: 498), DOM7r-15 (SEQ ID NO: 499), DOM7r-16 (SEQ ID NO: 500), DOM7r-17 (SEQ ID NO: 501), DOM7r-18 (SEQ ID NO: 502), DOM7r-19 (SEQ ID NO: 503), DOM7r-20 (SEQ ID NO: 504), DOM7r-21 (SEQ ID NO: 505), DOM7r-22 (SEQ ID NO: 506), DOM7r-23 (SEQ ID NO: 507), DOM7r-24 (SEQ ID NO: 508), DOM7r-25 (SEQ ID NO: 509), DOM7r-26 (SEQ ID NO: 510), DOM7r-27 (SEQ ID NO: 511), DOM7r-28 (SEQ ID NO: 512), DOM7r-29 (SEQ ID NO: 513), DOM7r-30 (SEQ ID NO: 514), DOM7r-31 (SEQ ID NO: 515), DOM7r-32 (SEQ ID NO: 516), DOM7r-33 (SEQ ID NO: 517) (the SEQ ID No's in this paragraph are those that appear in WO2007080392),
dAb8, dAb 10, dAb36, dAb7r20, dAb7r21, dAb7r22, dAb7r23, dAb7r24, dAb7r25, dAb7r26, dAb7r27, dAb7r28, dAb7r29, dAb7r30, dAb7r31, dAb7r32, dAb7r33, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7m12, dAb7m16, dAb7m26, dAb7r1, dAb7r3, dAb7r4, dAb7r5, dAb7r7, dAb7r8, dAb7r13, dAb7r14, dAb7r15, dAb7r16, dAb7r17, dAb7r18, dAb7r19, dAb7h1, dAb7h2, dAb7h6, dAb7h7, dAb7h8, dAb7h9, dAb7h10, dAb7h11, dAb7h12, dAb7h13, dAb7h14, dAb7 p1, and dAb7p2.

For example, the dAb that binds human serum albumin can comprise an amino acid sequence that has at least about 90%, or at least about 95%, or at least about 96%, or at least about 97%, or at least about 98%, or at least about 99% amino acid sequence identity with DOM7h-2 (SEQ ID NO:482), DOM7h-3 (SEQ ID NO:483), DOM7h-4 (SEQ ID NO:484), DOM7h-6 (SEQ ID NO:485), DOM7h-1 (SEQ ID NO:486), DOM7h-7 (SEQ ID NO:487), DOM7h-8 (SEQ ID NO:496), DOM7r-13 (SEQ ID NO:497), DOM7r-14 (SEQ ID NO:498), DOM7h-22 (SEQ ID NO:489), DOM7h-23 (SEQ ID NO:490), DOM7h-24 (SEQ ID NO:491), DOM7h-25 (SEQ ID NO:492), DOM7h-26 (SEQ ID NO:493), DOM7h-21 (SEQ ID NO:494) or DOM7h-27 (SEQ ID NO:495) (the SEQ ID No's in this paragraph are those that appear in WO2007080392), or
dAb8, dAb 10, dAb36, dAb7h21, dAb7h22, dAb7h23, Ab7h24, Ab7h25, Ab7h26, dAb7h27, dAb7h30, dAb7h31, dAb2, dAb4, dAb7, dAb11, dAb12, dAb13, dAb15, dAb16, dAb17, dAb18, dAb19, dAb21, dAb22, dAb23, dAb24, dAb25, dAb26, dAb27, dAb30, dAb31, dAb33, dAb34, dAb35, dAb38, dAb41, dAb46, dAb47, dAb52, dAb53, dAb54, dAb55, dAb56, dAb7h1, dAb7h2, dAb7h6, dAb7h7, dAb7h8, dAb7h9, dAb7h10, dAb7h11, dAb7h12, dAb7h13 or dAb7h14.
In certain embodiments, the dAb binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with the amino acid sequence of a dAb selected from the group consisting of
DOM7h-2 (SEQ ID NO:482), DOM7h-6 (SEQ ID NO:485), DOM7h-1 (SEQ ID NO:486), DOM7h-7 (SEQ ID NO:487), DOM7h-8 (SEQ ID NO:496), DOM7h-22 (SEQ ID NO:489), DOM7h-23 (SEQ ID NO:490), DOM7h-24 (SEQ ID NO:491), DOM7h-25 (SEQ ID NO:492), DOM7h-26 (SEQ ID NO:493), DOM7h-21 (SEQ ID NO:494), DOM7h-27 (SEQ ID NO:495) (the SEQ ID No's in this paragraph are those that appear in WO2007080392),
dAb7h21, dAb7h22, dAb7h23, Ab7h24, Ab7h25, Ab7h26, dAb7h27, dAb7h30, dAb7h31, dAb2, dAb4, dAb7, dAb38, dAb41, dAb7h1, dAb7h2, dAb7h6, dAb7h7, dAb7h8, dAb7h9, dAb7h10, dAb7h11, dAb7h12, dAb7h13 and dAb7h14.

In more particular embodiments, the dAb is a V_{κ} dAb that binds human serum albumin and has an amino acid sequence selected from the group consisting of
DOM7h-2 (SEQ ID NO:482), DOM7h-6 (SEQ ID NO:485), DOM7h-1 (SEQ ID NO:486), DOM7h-7 (SEQ ID NO:487), DOM7h-8 (SEQ ID NO:496) (the SEQ ID No's in this paragraph are those that appear in WO2007080392),
dAb2, dAb4, dAb7, dAb38, dAb41, dAb54, dAb7h1, dAb7h2, dAb7h6, dAb7h7, dAb7h8, dAb7h9, dAb7h10, dAb7h11, dAb7h12, dAb7h13 and dAb7h14.

In more particular embodiments, the dAb is a V_{H} dAb that binds human serum albumin and has an amino acid sequence selected from dAb7h30 and dAb7h31.

In more particular embodiments, the dAb is dAb7h11 or dAb7h14.

In other embodiments, the dAb, ligand or antagonist binds human serum albumin and comprises one, two or three of the CDRs of any of the foregoing amino acid sequences, eg one, two or three of the CDRs of dAb7h11 or dAb7h14.

Suitable Camelid V_{HH} that bind serum albumin include those disclosed in WO 2004/041862 (Ablynx N.V.) and in WO2007080392 (which V_{HH} sequences and their nucleic acid counterpart are incorporated herein by reference and form part of the disclosure of the present text), such as Sequence A (SEQ ID NO:518), Sequence B (SEQ ID NO:519), Sequence C (SEQ ID NO:520), Sequence D (SEQ ID NO:521), Sequence E (SEQ ID NO:522), Sequence F (SEQ ID NO:523), Sequence G (SEQ ID NO:524), Sequence H (SEQ ID NO:525), Sequence I (SEQ ID NO:526), Sequence J (SEQ ID NO:527), Sequence K (SEQ ID NO:528), Sequence L (SEQ ID NO:529), Sequence M (SEQ ID NO:530), Sequence N (SEQ ID NO:531), Sequence O (SEQ ID NO:532), Sequence P (SEQ ID NO:533), Sequence Q (SEQ ID NO:534), these sequence numbers corresponding to those cited in WO2007080392 or WO 2004/041862 (Ablynx N.V.). In certain embodiments, the Camelid V_{HH} binds human serum albumin and comprises an amino acid sequence that has at least about 80%, or at least about 85%, or at least about 90%, or at least about 95%, or at least about 96%, or at least about 97%, or at least about 98%, or at least about 99% amino acid sequence identity with ALB1 disclosed in WO2007080392 or any one of SEQ ID NOS:518-534, these sequence numbers corresponding to those cited in WO2007080392 or WO 2004/041862.

In some embodiments, the ligand or antagonist comprises an anti-serum albumin dAb that competes with any anti-serum albumin dAb disclosed herein for binding to serum albumin (e.g, human serum albumin).

In an alternative embodiment, the antagonist or ligand comprises a binding moiety specific for IL-13 (e.g., human IL-13), wherein the moiety comprises non-immunoglobulin sequences as described in WO2008096158, the disclosure of these binding moieties, their methods of production and selection (e.g., from diverse libraries) and their sequences are incorporated herein by reference as part of the disclosure of the present text)

### Conjugation to a Half-Life Extending Moiety (e.g., Albumin)

In one embodiment, a (one or more) half-life extending moiety (e.g., albumin, transferrin and fragments and analogues thereof) is conjugated or associated with the IL-13-binding polypeptide, dAb or antagonist of the invention. Examples of suitable albumin, albumin fragments or albumin variants for use in a IL-13-binding format are described in WO 2005077042, which disclosure is incorporated herein by reference and forms part of the disclosure of the present text. In particular, the following albumin, albumin fragments or albumin variants can be used in the present invention:
SEQ ID NO:1 (as disclosed in WO 2005077042, this sequence being explicitly incorporated into the present disclosure by reference);
Albumin fragment or variant comprising or consisting of amino acids 1-387 of SEQ ID NO:1 in WO 2005077042;
Albumin, or fragment or variant thereof, comprising an amino acid sequence selected from the group consisting of: (a) amino acids 54 to 61 of SEQ ID NO:1 in WO 2005077042; (b) amino acids 76 to 89 of SEQ ID NO:1 in WO 2005077042; (c) amino acids 92 to 100 of SEQ ID NO:1 in WO 2005077042; (d) amino acids 170 to 176 of SEQ ID NO:1 in WO 2005077042; (e) amino acids 247 to 252 of SEQ ID NO:1 in WO 2005077042; (f) amino acids 266 to 277 of SEQ ID NO:1 in WO 2005077042; (g) amino acids 280 to 288 of SEQ ID NO:1 in WO 2005077042; (h) amino acids 362 to 368 of SEQ ID NO:1 in WO 2005077042; (i) amino acids 439 to 447 of SEQ ID NO:1 in WO 2005077042 (j) amino acids 462 to 475 of SEQ ID NO:1 in WO 2005077042; (k) amino acids 478 to 486 of SEQ ID NO:1 in WO 2005077042; and (I) amino acids 560 to 566 of SEQ ID NO:1 in WO 2005077042.

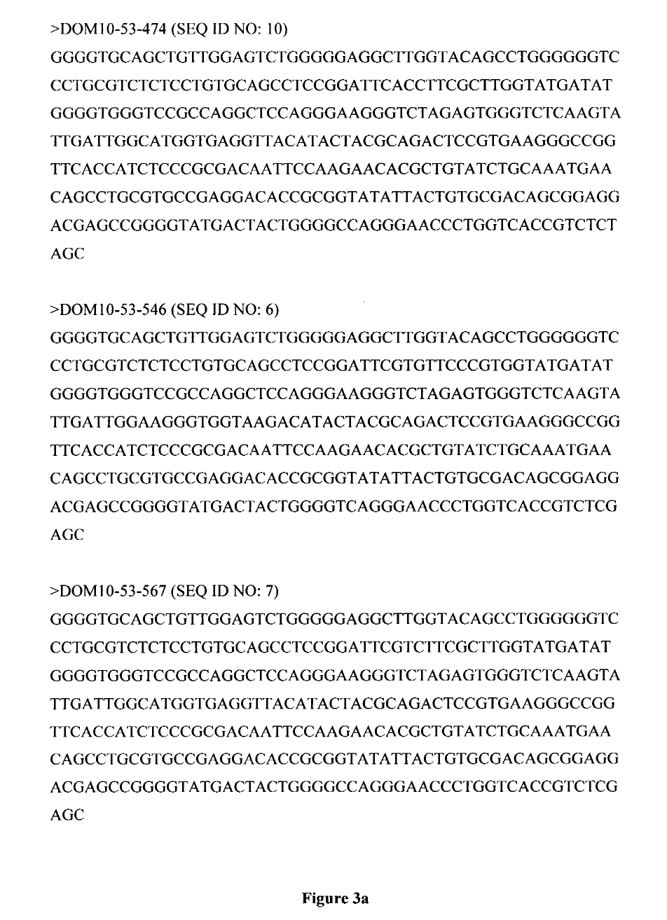

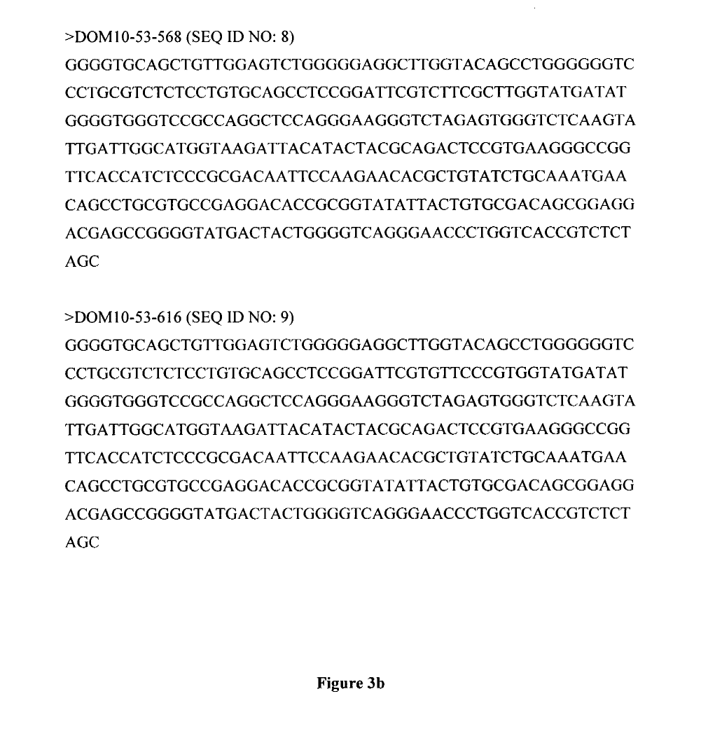

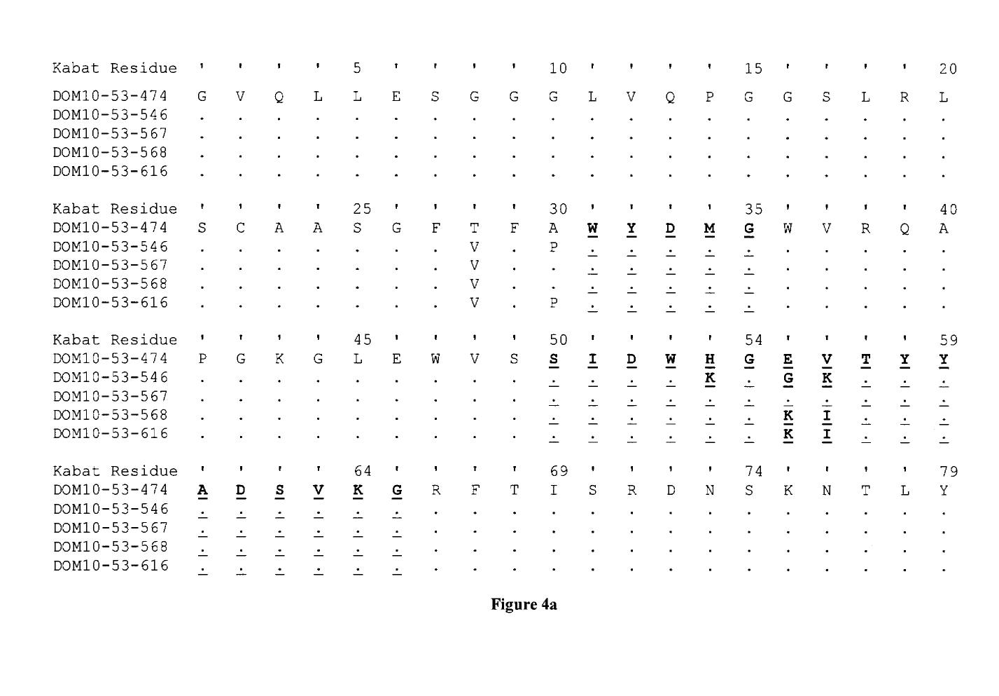

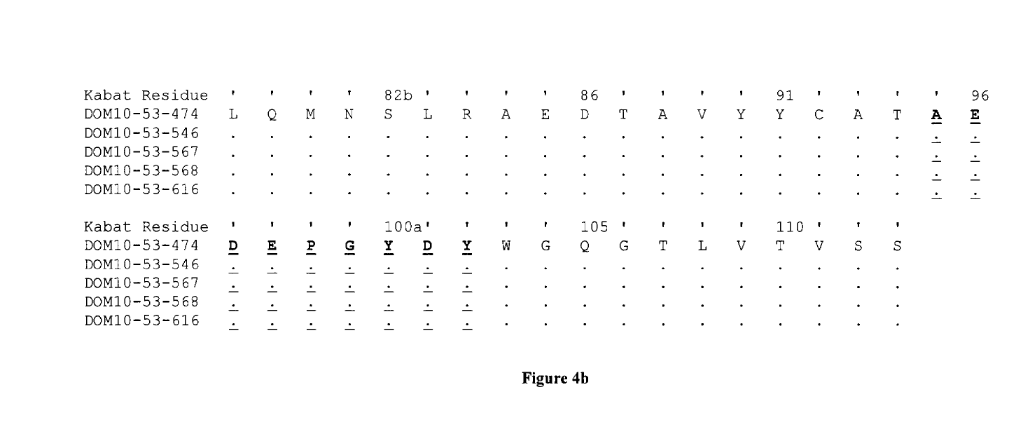

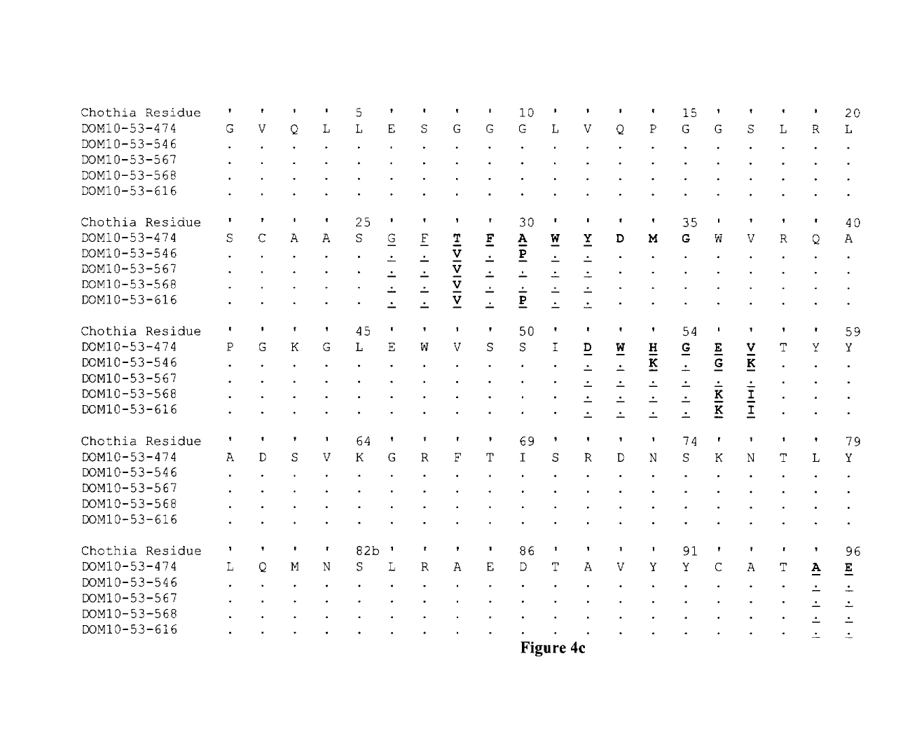

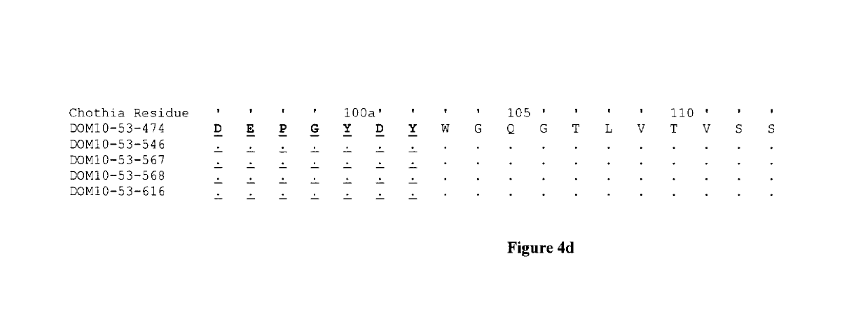

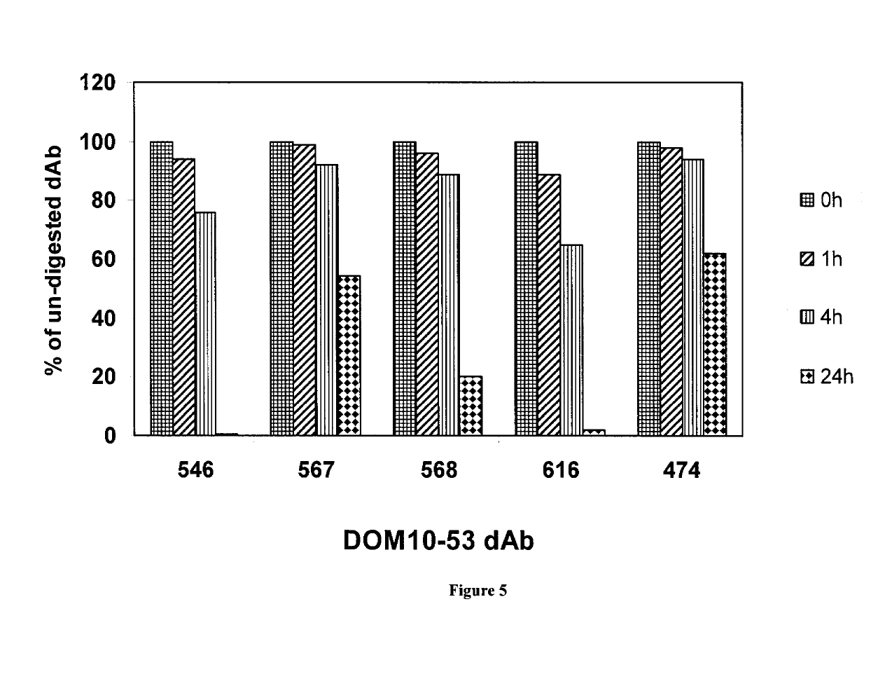

Further examples of suitable albumin, fragments and analogs for use in a IL-13-binding format are described in WO 03076567, which disclosure is incorporated herein by reference and which forms part of the disclosure of the present text. In particular, the following albumin, fragments or variants can be used in the present invention:
Human serum albumin as described in WO 03076567, e.g., in FIG. 3 (this sequence information being explicitly incorporated into the present disclosure by reference);
Human serum albumin (HA) consisting of a single non-glycosylated polypeptide chain of 585 amino acids with a formula molecular weight of 66,500 (See, Meloun, et al., FEBS Letters 58:136 (1975); Behrens, et al., Fed. Proc. 34:591 (1975); Lawn, et al., Nucleic Acids Research 9:6102-6114 (1981); Minghetti, et al., J. Biol. Chem. 261:6747 (1986));
A polymorphic variant or analog or fragment of albumin as described in Weitkamp, et al., Ann. Hum. Genet. 37:219 (1973);
An albumin fragment or variant as described in EP 322094, e.g., HA (1-373, HA (1-388), HA (1-389), HA (1-369), and HA (1-419) and fragments between 1-369 and 1-419;
An albumin fragment or variant as described in EP 399666, e.g., HA (1-177) and HA (1-200) and fragments between HA (1-X), where X is any number from 178 to 199.

Where a (one or more) half-life extending moiety (e.g., albumin, transferrin and fragments and analogues thereof) is used to format the IL-13-binding polypeptides, dAbs and antagonists of the invention, it can be conjugated using any suitable method, such as, by direct fusion to the IL-13-binding moiety (e.g., anti-IL-13dAb), for example by using a single nucleotide construct that encodes a fusion protein, wherein the fusion protein is encoded as a single polypeptide chain with the half-life extending moiety located N- or C-terminally to the IL-13 binding moiety. Alternatively, conjugation can be achieved by using a peptide linker between moieties, e.g., a peptide linker as described in WO 03076567 or WO 2004003019 (these linker disclosures being incorporated by reference in the present disclosure to provide examples for use in the present invention). Typically, a polypeptide that enhances serum half-life in vivo is a polypeptide which occurs naturally in vivo and which resists degradation or removal by endogenous mechanisms which remove unwanted material from the organism (e.g, human). For example, a polypeptide that enhances serum half-life in vivo can be selected from proteins from the extracellular matrix, proteins found in blood, proteins found at the blood brain barrier or in neural tissue, proteins localized to the kidney, liver, lung, heart, skin or bone, stress proteins, disease-specific proteins, or proteins involved in Fc transport.

In embodiments of the invention described throughout this disclosure, instead of the use of an anti-IL-13 “dAb” in an antagonist or ligand of the invention, it is contemplated that the skilled addressee can use a polypeptide or domain that comprises one or more or all 3 of the CDRs of a dAb of the invention that binds IL-13 (e.g, CDRs grafted onto a suitable protein scaffold or skeleton, eg an affibody, an SpA scaffold, an LDL receptor class A domain or an EGF domain) The disclosure as a whole is to be construed accordingly to provide disclosure of antagonists using such domains in place of a dAb. In this respect, see WO2008096158, the disclosure of which is incorporated by reference).

In one embodiment, therefore, an antagonist of the invention comprises an immunoglobulin single variable domain or domain antibody (dAb) that has binding specificity for IL-13 or the complementarity determining regions of such a dAb in a suitable format. The antagonist can be a polypeptide that consists of such a dAb, or consists essentially of such a dAb. The antagonist can be a polypeptide that comprises a dAb (or the CDRs of a dAb) in a suitable format, such as an antibody format (e.g, IgG-like format, scFv, Fab, Fab′, F(ab′)₂), or a dual specific ligand that comprises a dAb that binds IL-13 and a second dAb that binds another target protein, antigen or epitope (e.g, serum albumin).

Polypeptides, dAbs and antagonists according to the invention can be formatted as a variety of suitable antibody formats that are known in the art, such as, IgG-like formats, chimeric antibodies, humanized antibodies, human antibodies, single chain antibodies, bispecific antibodies, antibody heavy chains, antibody light chains, homodimers and heterodimers of antibody heavy chains and/or light chains, antigen-binding fragments of any of the foregoing (e.g, a Fv fragment (e.g, single chain Fv (scFv), a disulfide bonded Fv), a Fab fragment, a Fab′ fragment, a F(ab′)₂ fragment), a single variable domain (e.g, V₁₁, V_{L}), a dAb, and modified versions of any of the foregoing (e.g, modified by the covalent attachment of polyalkylene glycol (e.g, polyethylene glycol, polypropylene glycol, polybutylene glycol) or other suitable polymer).

In some embodiments, the invention provides a ligand (e.g., an anti-IL-13 antagonist) that is an IgG-like format. Such formats have the conventional four chain structure of an IgG molecule (2 heavy chains and two light chains), in which one or more of the variable regions (V_{H} and or V_{L}) have been replaced with a dAb of the invention. In one embodiment, each of the variable regions (2 V_{H} regions and 2 V_{L} regions) is replaced with a dAb or single variable domain, at least one of which is an anti-IL-13 dAb according to the invention. The dAb(s) or single variable domain(s) that are included in an IgG-like format can have the same specificity or different specificities. In some embodiments, the IgG-like format is tetravalent and can have one (anti-IL-13 only), two (e.g., anti-IL-13 and anti-SA), three or four specificities. For example, the IgG-like format can be monospecific and comprises 4 dAbs that have the same specificity; bispecific and comprises 3 dAbs that have the same specificity and another dAb that has a different specificity; bispecific and comprise two dAbs that have the same specificity and two dAbs that have a common but different specificity; trispecific and comprises first and second dAbs that have the same specificity, a third dAb with a different specificity and a fourth dAb with a different specificity from the first, second and third dAbs; or tetraspecific and comprise four dAbs that each have a different specificity. Antigen-binding fragments of IgG-like formats (e.g, Fab, F(ab′)₂, Fab′, Fv, scFᵥ) can be prepared. In one embodiment, the IgG-like formats or antigen-binding fragments may be monovalent for IL-13. If complement activation and/or antibody dependent cellular cytotoxicity (ADCC) function is desired, the ligand can be an IgG1-like format. If desired, the IgG-like format can comprise a mutated constant region (variant IgG heavy chain constant region) to minimize binding to Fc receptors and/or ability to fix complement. (see e.g, Winter et al, GB 2,209,757 B; Morrison et al., WO 89/07142; Morgan et al., WO 94/29351, Dec. 22, 1994).

The ligands of the invention (e.g., polypeptides, dAbs and antagonists) can be formatted as a fusion protein that contains a first immunoglobulin single variable domain that is fused directly to a second immunoglobulin single variable domain. If desired such a format can further comprise a half-life extending moiety. For example, the ligand can comprise a first immunoglobulin single variable domain that is fused directly to a second immunoglobulin single variable domain that is fused directly to an immunoglobulin single variable domain that binds serum albumin.

Generally the orientation of the polypeptide domains that have a binding site with binding specificity for a target, and whether the ligand comprises a linker, is a matter of design choice. However, some orientations, with or without linkers, may provide better binding characteristics than other orientations. All orientations (e.g, dAb1-linker-dAb2; dAb2-linker-dAb1) are encompassed by the invention are ligands that contain an orientation that provides desired binding characteristics can be easily identified by screening.

Polypeptides and dAbs according to the invention, including dAb monomers, dimers and trimers, can be linked to an antibody Fc region, comprising one or both of C_{H}2 and C_{H}3 domains, and optionally a hinge region. For example, vectors encoding ligands linked as a single nucleotide sequence to an Fc region may be used to prepare such polypeptides.

The invention moreover provides dimers, trimers and polymers of the aforementioned dAb monomers.

Ligands that Contain a Toxin Moiety or Toxin

The invention also relates to ligands (e.g., anti-IL-13 dAb, dAb monomer) that comprise a toxin moiety or toxin. Suitable toxin moieties comprise a toxin (e.g, surface active toxin, cytotoxin). The toxin moiety or toxin can be linked or conjugated to the ligand using any suitable method. For example, the toxin moiety or toxin can be covalently bonded to the ligand directly or through a suitable linker. Suitable linkers can include noncleavable or cleavable linkers, for example, pH cleavable linkers that comprise a cleavage site for a cellular enzyme (e.g, cellular esterases, cellular proteases such as cathepsin B). Such cleavable linkers can be used to prepare a ligand that can release a toxin moiety or toxin after the ligand is internalized.

A variety of methods for linking or conjugating a toxin moiety or toxin to a ligand can be used. The particular method selected will depend on the toxin moiety or toxin and ligand to be linked or conjugated. If desired, linkers that contain terminal functional groups can be used to link the ligand and toxin moiety or toxin. Generally, conjugation is accomplished by reacting toxin moiety or toxin that contains a reactive functional group (or is modified to contain a reactive functional group) with a linker or directly with a ligand. Covalent bonds formed by reacting a toxin moiety or toxin that contains (or is modified to contain) a chemical moiety or functional group that can, under appropriate conditions, react with a second chemical group thereby forming a covalent bond. If desired, a suitable reactive chemical group can be added to ligand or to a linker using any suitable method. (See, e.g, Hermanson, G. T., Bioconjugate Techniques, Academic Press: San Diego, Calif. (1996).) Many suitable reactive chemical group combinations are known in the art, for example an amine group can react with an electrophilic group such as tosylate, mesylate, halo (chloro, bromo, fluoro, iodo), N-hydroxysuccinimidyl ester (NHS), and the like. Thiols can react with maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

Suitable toxin moieties and toxins include, for example, a maytansinoid (e.g, maytansinol, e.g, DM1, DM4), a taxane, a calicheamicin, a duocarmycin, or derivatives thereof. The maytansinoid can be, for example, maytansinol or a maytansinol analogue. Examples of maytansinol analogs include those having a modified aromatic ring (e.g, C-19-decloro, C-20-demethoxy, C-20-acyloxy) and those having modifications at other positions (e.g, C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogs are described, for example, in U.S. Pat. Nos. 5,208,020 and 6,333,410, the contents of which are incorporated herein by reference. Maytansinol can be coupled to antibodies and antibody fragments using, e.g, an N-succinimidyl 3-(2-pyridyldithio)proprionate (also known as N-succinimidyl 4-(2-pyridyldithio)pentanoate (or SPP), 4-succinimidyl-oxycarbonyl-a-(2-pyridyldithio)-toluene (SMPT), N-succinimidyl-3-(2-pyridyldithio)butyrate (SDPB), 2 iminothiolane, or S-acetylsuccinic anhydride. The taxane can be, for example, a taxol, taxotere, or novel taxane (see, e.g, WO 01/38318). The calicheamicin can be, for example, a bromo-complex calicheamicin (e.g, an alpha, beta or gamma bromo-complex), an iodo-complex calicheamicin (e.g, an alpha, beta or gamma iodo-complex), or analogs and mimics thereof. Bromo-complex calicheamicins include I1-BR, I2-BR, I3-BR, I4-BR, J1-BR, J2-BR and K1-BR. Iodo-complex calicheamicins include I1-1, I2-1, I3-1, J1-1, J2-1, L1-1 and K1-BR. Calicheamicin and mutants, analogs and mimics thereof are described, for example, in U.S. Pat. Nos. 4,970,198; 5,264,586; 5,550,246; 5,712,374, and 5,714,586, the contents of each of which are incorporated herein by reference. Duocarmycin analogs (e.g, KW-2189, DC88, DC89 CBI-TMI, and derivatives thereof are described, for example, in U.S. Pat. No. 5,070,092, U.S. Pat. No. 5,187,186, U.S. Pat. No. 5,641,780, U.S. Pat. No. 5,641,780, U.S. Pat. No. 4,923,990, and U.S. Pat. No. 5,101,038, the contents of each of which are incorporated herein by reference.

Examples of other toxins include, but are not limited to antimetabolites (e.g, methotrexate, 6-mercaptopurine, 6-thioguanine, cytarabine, 5-fluorouracil decarbazine), alkylating agents (e.g, mechlorethamine, thioepa chlorambucil, CC-1065 (see U.S. Pat. Nos. 5,475,092, 5,585,499, 5,846,545), melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g, daunorubicin (formerly daunomycin) and doxorubicin), antibiotics (e.g, dactinomycin (formerly actinomycin), bleomycin, mithramycin, mitomycin, puromycin anthramycin (AMC)), duocarmycin and analogs or derivatives thereof, and anti-mitotic agents (e.g, vincristine, vinblastine, taxol, auristatins (e.g, auristatin E) and maytansinoids, and analogs or homologs thereof.

The toxin can also be a surface active toxin, such as a toxin that is a free radical generator (e.g, selenium containing toxin moieties), or radionuclide containing moiety. Suitable radionuclide containing moieties, include for example, moieties that contain radioactive iodine (¹³¹I or ¹²⁵I), yttrium (⁹⁰Y), lutetium (⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga).

The toxin can be a protein, polypeptide or peptide, from bacterial sources, e.g, diphtheria toxin, pseudomonas exotoxin (PE) and plant proteins, e.g, the A chain of ricin (RTA), the ribosome inactivating proteins (RIPs) gelonin, pokeweed antiviral protein, saporin, and dodecandron are contemplated for use as toxins.

Antisense compounds of nucleic acids designed to bind, disable, promote degradation or prevent the production of the mRNA responsible for generating a particular target protein can also be used as a toxin. Antisense compounds include antisense RNA or DNA, single or double stranded, oligonucleotides, or their analogs, which can hybridize specifically to individual mRNA species and prevent transcription and/or RNA processing of the mRNA species and/or translation of the encoded polypeptide and thereby effect a reduction in the amount of the respective encoded polypeptide. Ching, et al., Proc. Natl. Acad. Sci. U.S.A. 86: 10006-10010 (1989); Broder, et al., Ann. Int. Med. 113: 604-618 (1990); Loreau, et al., FEBS Letters 274: 53-56 (1990); Useful antisense therapeutics include for example: Veglin™ (VasGene) and OGX-011 (Oncogenix).

Toxins can also be photoactive agents. Suitable photoactive agents include porphyrin-based materials such as porfimer sodium, the green porphyrins, chlorin E6, hematoporphyrin derivative itself, phthalocyanines, etiopurpurins, texaphrin, and the like.

The toxin can be an antibody or antibody fragment that binds an intracellular target, such as a dAb that binds an intracellular target (an intrabody). Such antibodies or antibody fragments (dAbs) can be directed to defined subcellular compartments or targets. For example, the antibodies or antibody fragments (dAbs) can bind an intracellular target selected from erbB2, EGFR, BCR-ABL, p21 Ras, Caspase3, Caspase7, Bcl-2, p53, Cyclin E, ATF-1/CREB, HPV16 E7, HP1, Type IV collagenases, cathepsin L as well as others described in Kontermann, R. E., Methods, 34:163-170 (2004), incorporated herein by reference in its entirety.

The examples of WO2007085815 are incorporated herein by reference to provide details of relevant assays, formatting and experiments that can be equally applied to ligands of the present invention.

### Assays

### IL-13

### IL-13 Sandwich ELISA

A MAXISORP™ plate (high protein binding ELISA plate, Nunc, Denmark) is coated overnight with 2.5 μg/ml coating antibody (Module Set, Bender MedSystems, Vienna, Austria), then washed once with 0.05% (v/v) Tween 20 in PBS before blocking with 0.5% (w/v) BSA 0.05% (v/v) Tween 20 in PBS. The plates are washed again before the addition of 150 pg/ml IL-13 (GSK) mixed with a dilution series of DOM10 dAb (i.e., an anti-IL-13 dAb) or IL-13 alone. The plates are washed twice before binding of IL-13 to the capture antibody is detected using biotin conjugated detection antibody (Module Set, Bender Medsystems), followed by peroxidase labelled Streptavidin (Module Set, Bender MedSystems). Finally the plate is washed three times then incubated with TMB substrate (KPL, Gaithersburg, USA), and the reaction stopped by the addition of HCl and the absorbance read at 450 nm. Anti-IL-13 dAb activity causes a decrease in IL-13 binding and therefore a decrease in absorbance compared with the IL-13 only control.

### IL-13 Receptor Binding Assay (RBA) Using the 8200 Cellular Detection System

SPHERO™ goat anti-human IgG (H&L) polystyrene particles (0.5% w/v) (goat-anti-human particles, Spherotech, Libertyville, USA) is coated overnight with 20 μg IL-13R alpha 1/Fc chimera or IL-13R alpha 2/Fc chimera (R&D Systems, Minneapolis, USA). The following reagents are then combined in a 384-well black sided clear bottomed FMAT plate (Applied Biosystems, Foster City, USA): dilution series of DOM-10 dAb or 0.1% (w/v) BSA in PBS; 0.5 pg/ml biotinylated anti-IL-13 antibody (R&D Systems); 0.25 μg/ml STREPTAVIDIN ALEXA FLUOR® 647 conjugate (fluorescent probe, Molecular Probes, Invitrogen Ltd, Paisley, UK); 10 ng/ml recombinant human IL-13 (R&D Systems); and 1:10 dilution of IL-13R2/Fc coated particles. The plate is incubated for seven hours before being read in the 8200 cellular detection system (Applied Biosystems). Binding of IL-13 to the receptor coated particle causes a complex to form which is detected as a fluorescent event by the 8200. Anti-IL-13 dAb activity causes a decrease in IL-13 binding and thus a decrease in fluorescent events compared with the IL-13 only control.

### IL-13 Cell Assay

Isolated dAbs can be tested for their ability to inhibit IL-13 induced proliferation in cultured TF-1 cells (ATCC® catalogue no. CRL-2003). Briefly, 40000 TF-1 cells in phenol red free RPMI media (Gibco, Invitrogen Ltd, Paisley, UK) are placed in the well of a tissue culture microtitre plate and mixed with 5 ng/ml final concentration IL-13 (R&D Systems, Minneapolis, USA) and a dilution of the dAb to be tested. The mixture is incubated for 72 hours at 37° C. 5% CO₂. CELLTITER 96® reagent (colorometric reagent for determining viability, Promega, Madison, USA) is then added and the number of cells per well is quantified by measuring the absorbance at 490 nm. Anti-IL-13 dAb activity causes a decrease in cell proliferation and a corresponding lower A₄₉₀ than IL-13 alone.

### BIACORE® Off-Rate Screening

A streptavidin coated SA chip (Biacore) is coated with approximately 500 RU of biotinylated IL-13 (R&D Systems, Minneapolis, USA). Supernatant containing soluble dAb is diluted 1:5 in running buffer. 50 to 100 μl of the diluted supernatant is injected (kininject) at 50 μl/min flow rate, followed by a 5 minute dissociation phase. Clones with improved off-rates compared to parent are identified by eye, or by measurement using BIAevaluation software v4.1 (Biacore).

Competition BIACORE® with anti IL-13 dAbs

These experiments can be performed on a BIACORE® 3000 instrument (surface plasmon resonance instrument, Biacore), using a streptavidin coated SA chip (Biacore) coupled with ˜400 RU of biotinylated IL-13 (R&D Systems). Analytes are passed over the antigen-coated flow-cell, with in-line referencing against a blank flow-cell, at a flow rate of 30 μl/min in HBS-EP running buffer (Biacore). The first dAb is injected, followed immediately by injection of the second dAb, using the Biacore's co-inject function. This competition protocol can generally be used to assess competition of a test antibody or fragment with a known dAb (or other antibody polypeptide) for binding to IL-13.

### Competition and Epitope Mapping

Epitope Mapping of anti IL-13 dAbs

To determine the epitope specificity of the anti-IL-13 dAbs, Biacore competition experiments can be performed. dAb is injected, followed immediately by injection of a second dAb. If one (the second) dAb does not bind to IL-13 to which the other (first) dAb, this indicates that these dAbs bind to the same epitope. This competition protocol can generally be used to assess competition (and epitope mapping) of a test antibody or fragment with a known dAb (or other antibody polypeptide) for binding to IL-13. A slightly modified BIAcore protocol is possible in which first dAbs are injected over an IL-13 surface, then a high affinity binding dAb is injected at high concentration (5 μM) saturating the IL-13 surface and finally the dAbs are again injected. If there is a difference between binding prior and post saturation with the high affinity dAb, the epitopes are at least partially overlapping.

### IL-13 Induced B Cell Proliferation

Blood can be collected from normal blood donors. PBMC are isolated using Ficoll gradient. B cells are then isolated using a negative B cell isolation kit (EasySep Negative isolation kit, Stem Cell Technologies Inc). Purity (optionally in excess of 98%) can be determined by flowcytometry and staining with CD3, CD4, CD8, CD14, CD19 and CD23. B cells are then plated at 1×10⁵ cells/well in the presence of IL-13 (10 ng/ml) in plates coated with irradiated CD40L⁺ L cells. Cultures are incubated for 5 days with the addition of 3[H]thymidine for the final 18 hours. Anti-IL-13 dAbs are added at the start of the culture at 10 or 100 nM.

### B Cell Proliferation Assay

It has been shown previously that CD40L is able to activate cells to be responsive to IL-13. Indeed donors can be tested to show a dose-dependent proliferation when their B cells were incubated with irradiated CD40L⁺ L cells and increasing concentrations of IL-13. As negative controls B cells alone or CD40L transfected L cells alone can be used. The addition of anti-IL-13 dAbs can be assessed to see if this results in an inhibition of IL-13 induced proliferation of B cells from donors.

By way of illustration, for inhibitory dAbs (see WO2007085815) the average inhibition was 80% and 100% at concentrations of 10 nM and 100 nM respectively. Complete inhibition of the B cell proliferation was also observed with 3 μg/ml of positive control anti-IL 13 mAb (R&D). Control dAbs that did not bind IL-13 failed to inhibit this B cell proliferation.

### Inhibition of IL-13 Binding to IL-13Rα2

Anti-IL-13 dAbs can be tested for their ability to inhibit binding of IL-13 to IL13Rα2 in a competition assay.

### Binding to Variant IL-13 (R130Q)

Genetic variants of IL-13 have been associated with an increased risk for asthma (Heinzmann et al. Hum Mol. Genet. (2000) 9549-59) and bronchial hyperresponsiveness (Howard et al., Am. J. Resp. Cell Molec. Biol. (2001) 377-384). Therefore to determine whether anti-IL-13 dAbs are able bind variant IL-13 (R130Q), the TF-1 proliferation assay can be performed with variant IL-13 (R130Q), and increasing amounts of dAb. dAbs that bind the variant would be able to inhibit variant IL-13 induced TF-1 proliferation with ND50, eg with values of approximately 0.5 to 10 nM.

Cross-Reactivity with Rhesus and Cynomolgous IL-13.

A desired requirement of dAbs would be cross-reactivity with rhesus and Cynomolgous IL-13. To that end, dAbs can be tested in the TF-1 cell proliferation assay in which cells are stimulated with human IL-13 (5 ng/ml, Peprotech), rhesus IL-13 (5 ng/ml, R&D systems) or cynomolgous IL-13 (1:4000 dilution of supernatant containing in-house expressed cynomolgous IL-13). A dose-response of the dAb will determine the ND50 in this set up.

### IL-4

### IL-4 Receptor Binding Assay (RBA)

A MaxiSorp™ plate (high protein binding ELISA plate, Nunc, Denmark) is coated overnight with 0.5 pg/ml recombinant human IL-4R/Fc (R&D Systems, Minneapolis, USA). The wells is washed three times with 0.1% (v/v) Tween 20 in PBS, followed by three washes with PBS, before blocking with 2% (w/v) BSA in PBS. The plates are washed again before the addition of 10 ng/ml biotinylated-IL-4 (R&D Systems) mixed with a dilution series of anti-IL-4 dAbs or IL-4. IL-4 binding was detected with peroxidase labelled anti-biotin antibody (Stratech, Soham, UK) and then developed with TBM substrate (KPL, Gaithersburg, USA). The reaction is stopped by the addition of HCl and the absorbance read at 450 nm. Anti-IL-4 dAb activity causes a decrease in IL-4 binding to the receptor and therefore a decrease in absorbance compared with the IL-4 only control.

### IL-4 Cell Assay

Isolated dAbs can be tested for their ability to inhibit IL-4 induced proliferation in cultured TF-1 cells (ATCC® catalogue no. CRL-2003). Briefly, 40000 TF-1 cells in phenol red free RPMI media (Gibco, Invitrogen Ltd, Paisley, UK) are placed in the well of a tissue culture microtitre plate and mixed with 1 ng/ml final concentration IL-4 (R&D Systems, Minneapolis, USA) and a dilution of the dAb to be tested. The mixture is incubated for 72 hours at 37° C. 5% CO₂. CellTiter 96® reagent (colorometric reagent for determining viability, Promega, Madison, USA) is then added and the number of cells per well was quantified by measuring the absorbance at 490 nm. Anti-IL-4 dAb activity causes a decrease in cell proliferation and a corresponding lower A₄₉₀ than IL-4 alone.

Competition Biacore® with anti IL-4 dAbs

These experiments can be performed on a Biacore® 3000 instrument, using a streptavidin coated SA chip (surface plasmon resonance system, Biacore) coupled with ˜400 RU of biotinylated IL-4 (R&D Systems). Analytes are passed over the antigen-coated flow-cell, with in-line referencing against a blank flow-cell, at a flow rate of 30 μl/min in HBS-EP running buffer (Biacore). The first dAb is injected, followed immediately by injection of the second dAb using the Biacore's co-inject function. This competition protocol can generally be used to assess competition of a test antibody or fragment with a known dAb (or other antibody polypeptide) for binding to IL-4.

### EXAMPLES

### Example 1

### Selection of DOM10 Lead dAbs

### DOM10-53-546

DOM10-53-546, an anti-IL-13 domain antibody (dAb) was isolated from in-line fusion libraries selected against human IL-13 as an attempt to isolate a dual targeting molecule for IL-4 and IL-13. IL-4 binding dAb DOM9-112-210 was linked with IL-13 binding dAb DOM10-53-409 using ASTKGPS linker to make DOM9-112-210-ASTKGPS-DOM10-53-409. DOM9-112-210 and DOM10-53-409 are disclosed in WO2007085815. ASTKGPS linker is disclosed in WO2007085814. DOM9 dAb was kept constant and twelve libraries of DOM10-53-409 were made each diversifying three residues of DOM10-53-409 spanning all three CDRs and framework residues surrounding CDRs, to select for in-line fusions with better potency and expression. These libraries were generated using oligonucleotides incorporating NNS codons to diversify the residues. NNS codons provide randomisation of the targeted residue by substituting with any of the 20 amino acids or single stop codon within a total of 32 codons. N represents one of all four nucleotides A, T, G and C and S represents G or C. Primary PCRs carried out using these oligonucleotides were then assembled using assembly PCR. Assembly PCR (also known as ‘pull-through’ or SOE (Splicing by Overlap Extension) PCR) allows the two primary PCR products (one diversified, one constant) to be brought together without digest or ligation, making use of the complementary ends of the two Primary PCR products.

In-line fusion libraries were subjected to 2 rounds of selections with streptavidin-coated magnetic beads (Dynal, Norway) and 10 nM biotinylated human IL-13. The IL-13 was biotinylated using a five fold molar excess of EZ-Link Sulfo-NHS-LC-Biotin reagent (Pierce, Rockford, USA) (Henderikx et al., 2002, Selection of antibodies against biotinylated antigens. Antibody Phage Display: Methods and protocols, Ed. O'Brien and Atkin, Humana Press).

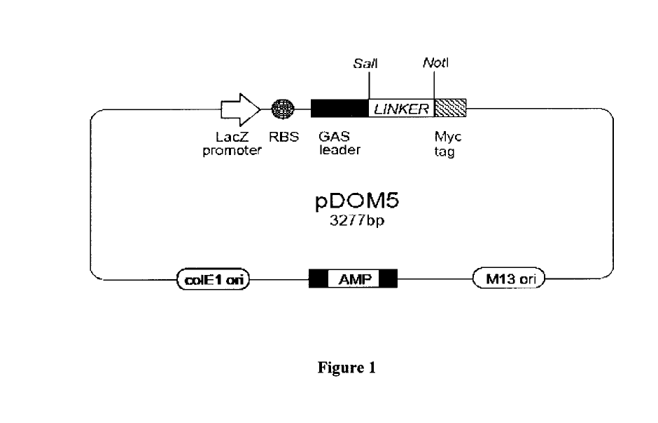

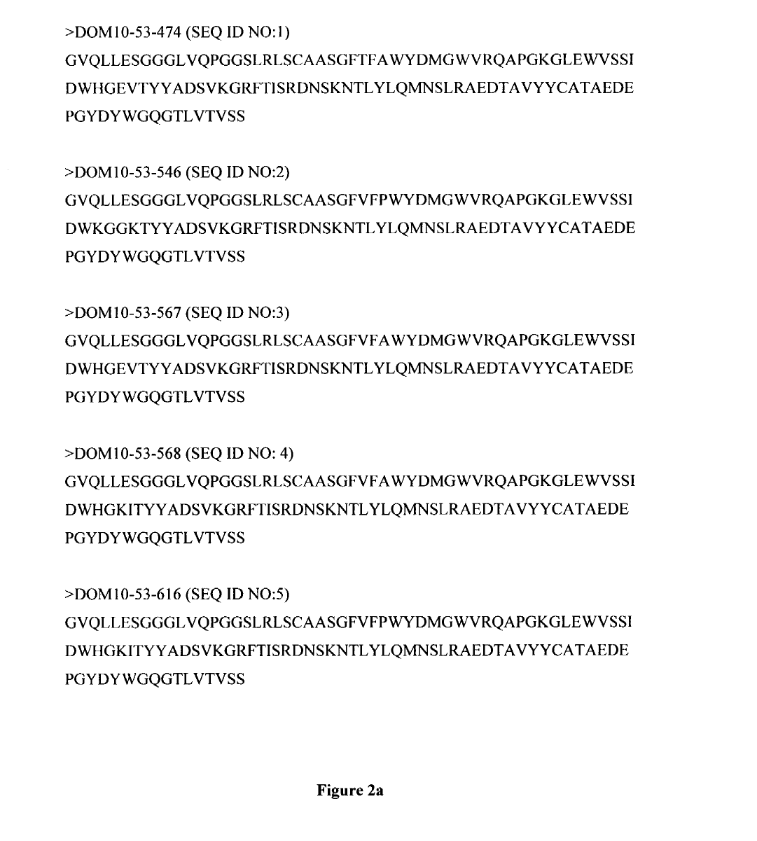

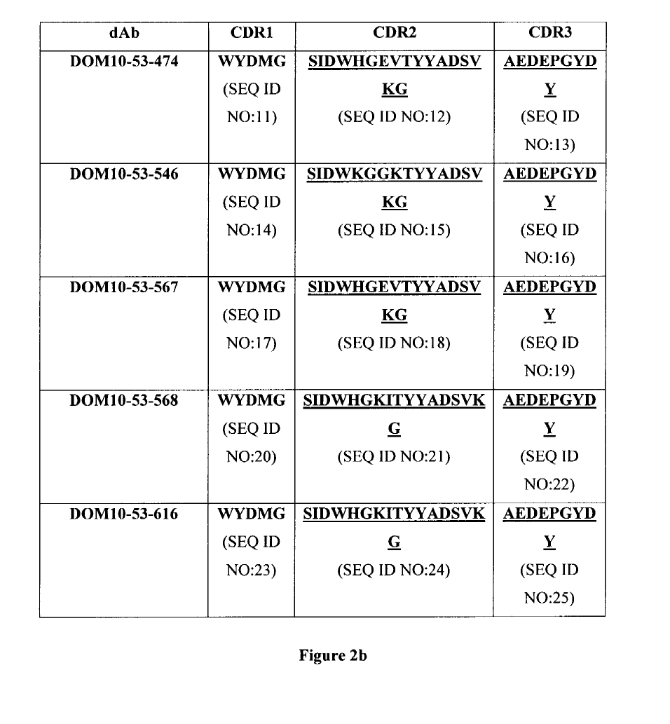

2^{nd} round outputs were cloned into pDOM5 vector (FIG. 1). pDOM5 is a pUC119-based expression vector under control of the LacZ promoter. Expression of dAbs into the supernatant was ensured by fusion to the universal GAS leader signal peptide (see WO2005093074) at the N-terminal end. In addition, a c-myc-tag was appended at the C-terminal end of the dAbs. After transformation of E. coli HB2151 cells, clones were expressed in 96 deep well plates in a high speed infors shaker programmed at 950 rpm, 30° C. and 80% humidity for 2 days in 0.5 ml/well overnight express auto-induction medium (high-level protein expression system, Novagen) supplemented with 100 μg/ml carbenicillin. Plates were then centrifuged at 4000 rpm for 20 minutes, supernatants were diluted 1/5 in HBS buffer and screened on Biacore™ for binding to 1500 Ru Protein A CMS chip to select for clones with improved expression. Protein A was coupled onto a CMS chip by primary amine coupling in accordance with the manufacturer's recommendations (Amine coupling kit, Biacore, GE healthcare]). Samples were run on Biacore at a flow rate of 50 μl/min. The surface was regenerated back to baseline using 0.1M Glycine pH 2. Any clones with improved expression were then expressed in 50 ml overnight express autoinduction medium at 30° C. for 48 hours and after cell pelleting by centrifugation (4,000 rpm for 20 min), the supernatants were incubated overnight at 4° C. with Streamline-protein A beads (Amersham Biosciences, binding capacity: 5 mg of dAb per mL of beads). The beads were then packed into drip columns, washed with 10 column volumes of PBS, and bound dAbs were eluted in 0.1 M glycine-HCl, pH 2.0. After neutralisation with 1 M Tris-HCl, pH 8.0, protein purity was estimated by visual analysis after SDS-PAGE on 12% acrylamide Tris-glycine gel (Invitrogen). Protein concentrations and yields (in mg per L of bacterial culture) were measured at 280 nm, using extinction coefficients calculated from the amino acid compositions. Clones with improved expression were analysed on Biacore for binding to 200Ru biotinylated human IL-13 streptavidin chip to assess the potency. The most potent clone selected from these libraries was DOM9-112-210-ASTKGPS-DOM10-53-546 (from the library where framework residues 28-30 were diversified) which demonstrated a similar expression level to DOM9-112-210-ASTKGPS-DOM10-53-409, but with improved potency for IL-13. The DOM10 arm, DOM10-53-546, was then cloned as a monomer into pDOM5 vector. DNA and amino acid sequences of DOM10-53-546 are summarised in FIGS. 2 and 3.

### DOM10-53-567

DOM10-53-567 was isolated from selections carried out on biotinylated cynomolgous (cyno) IL-13 of an error prone library of DOM10-53-474 (this dAb is disclosed in WO2007085815) in an attempt to select for dAbs with improved potency to cyno IL-13. The error prone library of DOM10-53-474 was made using GeneMorph PCR mutagenesis kit from Stratagene which utilises Mutazyme™ DNA polymerase according to manufacturer's instructions (Cat No 200550). 2 rounds of selections of DOM10-53-474 error prone library were performed with 10 nM and 5 nM cyno IL-13 respectively. 2^{nd} round outputs were cloned into pDOM5 vector, expressed in E. Coli HB2151 cells in 96 well deep well plates as explained above. Plates were centrifuged, supernatants were diluted 1/5 in HBS buffer and screened on 200Ru cyno IL-13 streptavidin chip on Biacore to look for clones with improved binding to cyno-IL-13 compared to DOM10-53-474. DOM10-53-567 was selected as a clone with improved binding kinetics to both cyno IL-13 and human IL-13. DNA and amino acid sequences are summarised in FIGS. 2 and 3.

### DOM10-53-568

In an attempt to further improve the potency of DOM10-53-567, the CDR2 of potent anti-IL-13 dAb DOM10-53-386 (this dAb is disclosed in WO2007085815) was introduced into DOM10-53-567 to generate DOM10-53-568 using assembly PCR and cloned into pDOM5 vector. DNA and amino acid sequences are summarised in FIGS. 2 and 3.

### DOM10-53-616

In an attempt to improve the potency of DOM10-53-546 further, CDR2 of potent dAb DOM10-53-386 was introduced to replace the CDR2 of DOM10-53-546 to generate DOM10-53-616 using assembly PCR and cloned into pDOM5 vector. DNA and amino acid sequences are summarised in FIGS. 2 and 3.

### Example 2

### Sequence Analysis

As shown in FIG. 4, DOM10-53-546 has amino acid changes at five positions compared to DOM10-53-474. Two framework mutations N terminus to CDR1 at positions 28 (T to V) and 30 (A to P), and three residues in CDR2 at positions 54 (H to K), 56 (E to G) and 57 (V to K). DOM10-53-567 is only one amino acid different from DOM10-53-474 at position 28 (T to V)

DOM10-53-568 also has this mutation but in addition it has two amino acid changes in CDR2 at positions 56 (E to K) and 57 (V to I). DOM 10-53-616 has all three mutations as DOM10-53-568 and in addition amino acid at position 30 is changed (A to P). Amino acid change from threonine to valine at position 28 is common to all four dAbs. All these dAbs including DOM10-53-474 has the same CDR1 and CDR3. All numberings are according to Kabat.

### Example 3

### Expression and Potency of DOM10 dAbs

As we demonstrate below, the new dAbs are more potent than DOM10-53-474. Furthermore, the new dAbs are species cross-reactive for binding (and potent IL-13 neutralisers cross species) between human and non-human primate IL-13. This makes the new dAbs very useful as drugs and as the basis for drug development to treat and/or prevent IL-13-mediated conditions and diseases, since such development usually entails testing of candidate leads in non-human primate species prior to testing in man, as the non-human primates are believed to be good models for humans and provide data to guide subsequent studies in man.

To do a side by side comparison of above mentioned anti-IL-13 DOM10 dAbs, they were cloned into pDOM5 vector without myc tag, transformed into Mach 1 chemical competent cells (Invitrogen) and expressed in 50 ml cultures in overnight express auto-induction medium as explained above. Protein was purified using streamline protein A as explained previously and purified protein was assessed by SDS PAGE. Briefly, for the SDS PAGE, 5 μl of dAb, 15 μl of H₂O and 6 μl of sample buffer were mixed and incubated at 90° C. for 10 minutes followed by 2 minutes on ice and 20 μl of sample was loaded into SDS PAGE gel. The results showed pure preparations of all the dAbs.

The expression levels of DOM10 dAbs are summarised in table 1. The expression levels of DOM10-53-546 and DOM10-53-616 were much better than that of DOM10-53-474 and the other new dAbs tested.

TABLE 1
Potency, expression and thermal stability data of DOM10 dAbs.

| | | | | | HEK | HEK | HEK | | Trypsin |
|---|---|---|---|---|---|---|---|---|---|
| | Biacore (KD) | | | | assay | assay | assay | | Stability |
| | IL-13 | | Expression | ELISA | Human | Cyno | Rhesus | | % active, |
| | Human | Cyno | In E. Coli | EC₅₀ | EC₅₀ | EC₅₀ | EC₅₀ | DSC | 24 h |
| dAb | (pM) | (nM) | (mg/L) | (nM) | (nM) | (nM) | (nM) | (° C.) | digestion |
| DOM10- | 259 | 9.3 | 3 | 0.217 | 2.0 | 30.1 | 30.2 | 53-57 | 62 |
| 53-474 | | | | | | | | | |
| DOM10- | 94 | 2.1 | 14 | 0.125 | 1.0 | 5.9 | 11.4 | 55.5 | 1 |
| 53-546 | | | | | | | | | |
| DOM10- | 72 | 2.6 | 3 | 0.021 | 0.31 | 5.1 | 2.8 | 54.1 | 52 |
| 53-567 | | | | | | | | | |
| DOM10- | 70 | 1.6 | 3 | — | 0.62 | 8.8 | 8.1 | 52.9 | 20 |
| 53-568 | | | | | | | | | |
| DOM10- | 130 | 3.2 | 11 | 0.048 | 2.9 | 5.9 | 7.6 | 49.9 | 2 |
| 53-616 | | | | | | | | | |

The binding affinities of purified DOM10 dAbs to both human and cyno IL-13 were assessed by Biacore analysis. Analysis was carried out using biotinylated IL-13. About 150 RU of biotinylated IL-13 was coated to a streptavidin (SA) chip (Biacore, GE healthcare). The surface was regenerated back to baseline using 0.1 M Glycine pH 2. dAbs were passed over this surface at defined concentrations using a flow rate of 50 μl/min. Data were analyzed using BIAevaluation software (Biacore, GE Healthcare) and fitted to the 1:1 model of binding. The binding data fitted well to the 1:1 model for all DOM10 dAbs. Biacore runs were carried out at 25° C. KD values for both human and cyno IL-13 dAbs are summarized in table 1. All of the new dAbs showed good potency against both human and cyno IL-13, and all were much better than DOM10-53-474. DOM10-53-567 and DOM10-53-568 demonstrated the best potency for both human and cyno IL-13.

dAbs were also tested in DOM10 ELISA to assess the potency against human IL-13 and cell based HEK Blue-STAT6 (HEK STAT) assays to assess the potency against human, cyno and Rhesus IL-13. DOM10 ELISA measures the ability of DOM10 dAbs to bind IL-13 and prevent its binding to an IL-13 detection antibody. A MAXISORP™ plate (high protein binding ELISA plate, Nunc, Denmark) is coated overnight with 2.5 μg/ml coating antibody (Module Set, Bender MedSystems, Vienna, Austria), then washed once with 0.1% (v/v) Tween 20 in PBS before blocking with 0.5% (w/v) BSA 0.05% (v/v) Tween 20 in PBS for 2 hours at room temperature. The plate is washed twice as previously detailed before the addition of 150 pg/ml IL-13 (GSK) mixed with a dilution series of DOM10 dAb (i.e., an anti-IL-13 dAb) and incubated for 1 hour. The plate is washed twice before binding of IL-13 to the capture antibody is detected using biotin conjugated detection antibody (Module Set, Bender Medsystems), followed by peroxidase labelled Streptavidin (Module Set, Bender MedSystems). Finally the plate is washed three times before incubation with TMB substrate (KPL, Gaithersburg, USA), and the reaction stopped by the addition of HCl and the absorbance read at 450 nm. Anti-IL-13 dAb activity causes a decrease in IL-13 binding and therefore a decrease in absorbance compared with the IL-13 only control.

HEK Blue-STAT6 assay measures the ability of dAbs to inhibit human IL-13 stimulated alkaline phosphatase production in HEKBlue-STAT6 cells in vitro. This assay uses HEK293 cells stably transfected with the STAT6 gene and the SEAP (secreted embryonic alkaline phosphatase) reporter gene (Invitrogen, San Diego). Upon stimulation with IL-13, SEAP is secreted into the supernatant which is measured using a colorimetric method. Soluble dAbs were tested for their ability to block IL-13 signalling via the STAT6 pathway.

Briefly, 5×10⁴ HEK-Blue STAT6 cells are cultured in DMEM (Gibco, Invitrogen Ltd, Paisley, UK) with dAb which has been pre-incubated for one hour at 37° C. with recombinant IL13 (GSK). The pre-incubation is performed using an equal volume of dAb and recombinant IL13. This pre-incubation mixture is then added to an equal volume of HEK-Blue STAT6 cells. Hence, the final concentration of IL13 in the assays is 3 ng/ml and the dAbs are tested in a dose response ranging between 200 nM and 0.05 nM. The plate is incubated for 24 hours at 37° C. 5% CO₂. The culture supernatant is then mixed with QuantiBlue (Invivogen) and the absorbance read at 640 nm. Anti-IL-13 dAb activity causes a decrease in STAT6 activation and a corresponding decrease in A₆₄₀ compared to IL-13 stimulation

Results of the assay data of DOM10 dAbs are summarized in table 1. In agreement with Biacore data, DOM10-53-567 demonstrated the best potency against human, cyno and Rhesus IL-13. Although DOM10-53-568 showed similar KD values to that of DOM10-53-567, it was less potent in the assays.

As shown by the data, all new dAbs are cross-reactive between human and cyno IL-13 and showed neutralization of all forms of IL-13 tested. All new dAbs (except DOM10-53-616) showed much more neutralizing potency for human, cyno and rhesus IL-13 than DOM10-53-474. DOM10-53-567 was a much more potent neutralizer than DOM10-53-474 for cyno and rhesus IL-13 and was broadly comparable against human IL-13.

### Example 4

### Protease Stability of new dAbs

Protease stability of the new dAbs were assessed using techniques as generally described in co-pending patent applications PCT/GB2008/050399 and PCT/GB2008/050405, the disclosures of which is incorporated herein in their entirety to provide details of methods of testing protease resistance of the dAbs, ligands and compositions of present invention, and to provide explicit disclosure herein of methods of using such protease-resistant dAbs of the invention.

To assess the trypsin stability of the present DOM10 clones, 0.3 mg/ml of purified protein was digested with sequencing grade trypsin (Promega) at 25:1 dAb:trypsin ratio in PBS buffer. Reaction was carried out at 30° C. for 0, 1, 4 and 24 h and reaction was ended by adding cocktail of protease inhibitors (Complete protease inhibitor tablets, Roche) and stored at −20° C. until further analysis.

Digested protein was diluted 1/400 in HBS buffer and passed over 150 Ru biotinylated human IL-13 streptavidin chip on Biacore at a flow rate of 50 μl/min to assess the amount of undigested dAb. The chip surface was regenerated with 10 ul 0.1 M glycine pH 2 between each injection cycle. Percentage of undigested dAb was calculated by comparing maximum Ru of undigested dAb with maximum Ru of digested dAb at each time point. Results are presented in FIG. 5. Out of the dAbs tested DOM10-53-567 was more resistant to trypsin cleavage compared to other dAbs and was more similar to that of DOM10-53-474. DOM10-53-546 and DOM10-53-616 were less resistant to trypsin cleavage compared to other dAbs. We believe that resistance to trypsin digestion represent stability of the dAb in vivo as it will be less prone to cleavage by proteases and hence longer half life.

### Example 5

### DSC (Differential Scanning Calorimetry)

The thermal stability of the dAbs was determined using differential scanning calorimeter (DSC). dAbs were tested at 1 mg/ml in PBS buffer. Proteins were dialysed overnight into PBS buffer. PBS buffer was used as a reference for all samples. DSC was performed using capillary cell microcalorimeter VP-DSC (Microcal, Mass., USA), at a heating rate of 180° C./hour. A typical scan usually was from 25-90° C. for both, the reference buffer and the protein sample. After each reference buffer and sample pair, the capillary cell was cleaned with a solution of 1% Decon (Fisher-Scientific) in water followed by PBS. Resulting data traces were analysed using Origin 7 Microcal software. The DSC trace obtained from the reference buffer was subtracted from the sample trace. The precise molar concentration of the sample was entered into the data analysis routine to yield values for melting temperature (Tm), enthalpy (ΔH) and van't Hoff enthalpy (ΔHv) values. Data were fitted to a non-2-state model. The results are summarized in table 1. The melting temperatures (Tm) of the dAbs ranged from 49.9° C.-55.5° C. DOM10-53-546 and DOM10-53-567 showed highest Tm values, 55.5° C. and 54.5° C. respectively. Among the dAbs tested, DOM10-53-567 not only benefit from having highest potency but also demonstrated reasonably high Tm, which is believed to indicate relatively high stability of the dAb. This would be beneficial as the dAb will be more stable at in vivo temperature maximising the efficacy and will have a good shelf life.

1. An anti-interleukin-13 (IL-13) immunoglobulin single variable domain comprising an amino acid sequence that is identical to DOM10-53-474, with the exception that the amino acid sequence has 1, 2, 3, 4 or 5 amino acid changes compared to DOM10-53-474 (SEQ ID NO: 1) and wherein the single variable domain has a valine at position 28 according to Kabat numbering, wherein the single variable domain is not DOM10-53-616 (SEQ ID NO: 5).

2. An anti-interleukin-13 (IL-13) immunoglobulin single variable domain comprising an amino acid sequence that is identical to DOM10-53-474, with the exception that the amino acid sequence has 1, 2, 3, 4 or 5 amino acid changes compared to DOM10-53-474 (SEQ ID NO: 1) and wherein the single variable domain has the sequence XGX′X″, wherein the G is at position 54 according to Kabat numbering and
X=H or K;
X′=G or K;
X″=K or I
wherein the single variable domain is not DOM10-53-616 (SEQ ID NO: 5).

3. The single variable domain of claim 2, wherein the variable domain has a valine at position 28 according to Kabat numbering.

4. The single variable domain of claim 1, wherein the variable domain has an amino acid change (versus DOM10-53-474 (SEQ ID NO: 1)) at one or more of position 30, 53, 55 and 56 (according to Kabat numbering).

5. The single variable domain of claim 1, wherein the variable domain has proline at position 30 (according to Kabat numbering).

6. The single variable domain of claim 1, wherein the variable domain has lysine at position 53 (according to Kabat numbering).

7. The single variable domain of claim 1, wherein the variable domain has glycine or lysine at position 55 (according to Kabat numbering).

8. The single variable domain of claim 1, wherein the variable domain has isoleucine or lysine at position 56 (according to Kabat numbering).

9. The single variable domain of claim 1, wherein the variable domain has lysine at position 55 and isoleucine at position 56 (according to Kabat numbering).

10. An anti-interleukin-13 (IL-13) immunoglobulin single variable domain, wherein the variable domain is DOM10-53-546 (SEQ ID NO: 2); DOM10-53-567 (SEQ ID NO: 3) or DOM10-53-568 (SEQ ID NO: 4).

11. An anti-interleukin-13 (IL-13) immunoglobulin single variable domain encoded by the nucleotide sequence of DOM10-53-546 (SEQ ID NO: 6); DOM10-53-567 (SEQ ID NO: 7) or DOM10-53-568 (SEQ ID NO: 8).

12. The single variable domain of claim 1, wherein the variable domain specifically binds human, cynomolgus monkey and rhesus IL-13.

13. The single variable domain of claim 1, wherein the variable domain neutralises human IL-13 in a standard HEK STAT assay with an EC₅₀ of 0.1 to 2.0 nM.

14. The single variable domain of claim 1, wherein the variable domain neutralises rhesus IL-13 in a standard HEK STAT assay with an EC₅₀ of 1 to 20 nM.

15. The single variable domain of claim 1, wherein the variable domain neutralises cynomolgus monkey IL-13 in a standard HEK STAT assay with an EC₅₀ of 1 to 20 nM.

16. An interleukin-13 (IL-13) antagonist comprising an anti-IL-13 immunoglobulin single variable domain according to claim 1.

17. The antagonist of claim 16, wherein the antagonist competes with DOM10-53-546 (SEQ ID NO: 2); DOM10-53-567 (SEQ ID NO: 3); DOM10-53-568 (SEQ ID NO: 4); or DOM10-53-616 (SEQ ID NO: 5) for binding to IL-13.

18. The antagonist of claim 16 or an interleukin-13 (IL-13) antagonist comprising DOM10-53-616 (SEQ ID NO: 5) for pulmonary delivery.

19. Use of the antagonist of claim 16 or an interleukin-13 (IL-13) antagonist comprising DOM10-53-616 (SEQ ID NO: 5) in the manufacture of a medicament for pulmonary delivery.

20. The antagonist of claim 16 for therapy or prophylaxis of an IL-13-mediated condition in a human.

21. Use of the antagonist of claim 16, in the manufacture of a medicament for therapy or prophylaxis of an IL-13-mediated condition in a human.

22. A method of treating and/or preventing an IL-13-mediated condition in a human patient, the method comprising administering the antagonist of claim 16 or an interleukin-13 (IL-13) antagonist comprising DOM10-53-616 (SEQ ID NO: 5) to the patient.

23. The antagonist of claim 20, the use of claim 21 or the method of claim 22, wherein the IL-13-mediated condition is a respiratory condition.

24. The antagonist, use or method of claim 23, wherein the IL-13-mediated condition is selected from lung inflammation, chronic obstructive pulmonary disease, asthma, pneumonia, hypersensitivity pneumonitis, pulmonary infiltrate with eosinophilia, environmental lung disease, pneumonia, bronchiectasis, cystic fibrosis, interstitial lung disease, primary pulmonary hypertension, pulmonary thromboembolism, disorders of the pleura, disorders of the mediastinum, disorders of the diaphragm, hypoventilation, hyperventilation, sleep apnea, acute respiratory distress syndrome, mesothelioma, sarcoma, graft rejection, graft versus host disease, lung cancer, allergic rhinitis, allergy, asbestosis, aspergilloma, aspergillosis, bronchiectasis, chronic bronchitis, emphysema, eosinophilic pneumonia, idiopathic pulmonary fibrosis, invasive pneumococcal disease, influenza, nontuberculous mycobacteria, pleural effusion, pneumoconiosis, pneumocytosis, pneumonia, pulmonary actinomycosis, pulmonary alveolar proteinosis, pulmonary anthrax, pulmonary edema, pulmonary embolus, pulmonary inflammation, pulmonary histiocytosis X, pulmonary hypertension, pulmonary nocardiosis, pulmonary tuberculosis, pulmonary veno-occlusive disease, rheumatoid lung disease, sarcoidosis, and Wegener's granulomatosis.

25. A pulmonary delivery device containing the antagonist of claim 16 or an interleukin-13 (IL-13) antagonist comprising DOM10-53-616 (SEQ ID NO: 5).

26. The device of claim 25, wherein the device is an inhaler or an intranasal administration device.

27. A dual specific ligand comprising a variable domain according to any one of claims 1.

28. An isolated or recombinant nucleic acid encoding a polypeptide comprising an immunoglobulin single variable domain according to any one of claims 1.

29. The nucleic acid according to claim 28, wherein the nucleic acid comprises the nucleotide sequence of DOM10-53-546 (SEQ ID NO: 6); DOM10-53-567 (SEQ ID NO: 7); DOM10-53-568 (SEQ ID NO: 8); or DOM10-53-616 (SEQ ID NO: 9).

30. An isolated or recombinant nucleic acid, wherein the nucleic acid comprises a nucleotide sequence that is at least 99% identical to the nucleotide sequence of DOM10-53-546 (SEQ ID NO: 6); DOM10-53-567 (SEQ ID NO: 7); DOM10-53-568 (SEQ ID NO: 8); or DOM10-53-616 (SEQ ID NO: 9), and wherein the nucleic acid encodes a polypeptide comprising an immunoglobulin single variable domain that specifically binds to IL-13.

31. A vector comprising the nucleic acid of claim 28, 29 or 30.

32. A host cell comprising the nucleic acid of claim 28, 29 or 30, or the vector of claim 31.

33. A method of producing polypeptide comprising an immunoglobulin single variable domain, the method comprising maintaining a host cell of claim 32 under conditions suitable for expression of said nucleic acid or vector, whereby a polypeptide comprising an immunoglobulin single variable domain is produced.

34. The method of claim 33, further comprising isolating the polypeptide and optionally producing a variant or a mutated variant, having an improved affinity and/or EC₅₀ for IL-13 neutralization in a standard HEK STAT assay than the isolated polypeptide.

35. A pharmaceutical composition comprising an immunoglobulin single variable domain of any one of claims 1 to 15 or an antagonist of any one of claim 16, 17, 18, 20, 23 or 24, and a pharmaceutically acceptable carrier, excipient or diluent.

36. A fusion protein comprising the single variable domain of any one of claims 1.

37. An isolated or recombinant nucleic acid encoding the fusion protein of claim 36.

38. The immunoglobulin single variable domain of any one of claims 1 to 15, or the antagonist of any one of claim 16, 17, 18, 20, 23 or 24, or the fusion protein of claim 36, comprising an antibody constant domain.

39. The variable domain, antagonist or fusion protein of claim 38, comprising an antibody Fc, optionally wherein the N-terminus of the Fc is linked (optionally directly linked) to the C-terminus of the variable domain.

40. The immunoglobulin single variable domain of any one of claims 1 to 15, or the antagonist of any one of claim 16, 17, 18, 20, 23 or 24, or the fusion protein of claim 36, wherein the variable domain wherein further comprises a half-life extending moiety.

41. The variable domain, antagonist or fusion protein of claim 40, wherein the half-life extending moiety is a polyethylene glycol moiety, serum albumin or a fragment thereof, transferrin receptor or a transferrin-binding portion thereof, or an antibody or antibody fragment comprising a binding site for a polypeptide that enhances half-life in vivo.

42. The variable domain, antagonist or fusion protein of claim 41, wherein the half-life extending moiety is an antibody or antibody fragment comprising a binding site for serum albumin or neonatal Fc receptor.

43. The variable domain, antagonist or fusion protein of claim 40, wherein the half-life extending moiety is a dAb, antibody or antibody fragment.
